SEQUENCE LISTING (21 sequences)

SEQ ID NO: 1 - DNA, 3273 bp, Artificial sequence
  Synthetic construct
atggcaacactcctcagtcatccacagcagagacctcctttcctcagacaggcaatcaaaatcagacggcggcgggtgcgggacctccaggatcccccaccccagatggccccagaaatccagccacccagccaccacttctcccccgaacagagggctctgctgtacgaggacgccctgtacaccgtgctgcacagactgggccacccagagcccaaccacgtgaccgaagccagcgagctgctgaggtacctgcaggaagctttccacgtggagccagaggaacaccagcagaccctgcagagagtgagagaactggagaagcccatcttctgcctgaaggctaccgtgaagcaggccaagggcatcctgggcaaggacgtgagcggcttctccgatccctactgcctgctgggcatcgagcagggggtgggggtgccaggcggcagccccggctccaggcacagacagaaggctgtcgtgaggcacaccatcccagaagaagaaacacacagaacccaggtgatcacccagaccctgaaccccgtgtgggacgaaaccttcatcctggaattcgaggacatcaccaatgccagcttccacctggacatgtgggacctggataccgtggaatccgtgaggcagaagctgggcgagctgaccgatctgcacggcctgaggagaatcttcaaggaagctaggaaggacaagggccaggacgatttcctgggcaacgtggtgctgagactgcaggacctgaggtgcagagaggatcagtggtacccactggaacccaggaccgaaacctacccagatagaggccagtgccacctgcagttccagctgatccacaagaggagagctaccagcgcctccagaagccagcccagctacaccgtgcacctgcacctgctgcagcagctggtgtcccacgaagtgacccagcacgaggctggatccaccagctgggatggctccctgagcccacaggctgctaccgtgctgttcctgcacgctacccagaaggacctgagcgatttccaccagtccatggcccagtggctggcttacagcaggctgtaccagtccctggagttcccaagctcctgcctgctgcaccccatcaccagcatcgaataccagtggatccagggcagactgaaggccgagcagcaggaagagctggccgcttccttcagctccctgctgacctacggcctgagcctgatcaggagattcaggtccgtgttcccactgtccgtgagcgattcccccgctaggctgcagagcctgctgagagtgctggtgcagatgtgcaagatgaaggctttcggcgaactgtgcccaaataccgccccactgccacagctggtgaccgaagctctgcagaccggcaccaccgagtggttccacctgaagcagcagcaccaccagccaatggtgcagggcatcccagaggctgggaaggctctgctgggcctggtgcaggacgtgatcggcgatctgcaccagtgccagaggacctgggacaagatcttccacaataccctgaagatccacctgttcagcatggccttcagggagctgcagtggctggtggctaagagagtgcaggaccacaccaccgtggtgggcgatgtggtgagcccagaaatgggcgagtccctgttccagctgtacatcagcctgaaggaactgtgccagctgaggatgagcagctccgagagagatggggtgctggctctggataacttccacaggtggttccagccagctatccccagctggctgcagaagacctacaatgaggctctggccagggtgcagagagccgtgcagatggatgaactggtgcccctgggcgagctgaccaagcacagcacctccgctgtggacctgagcacctgcttcgctcagatctcccacaccgctaggcagctggactggccagatccagaagaggccttcatgatcaccgtgaagttcgtggaagacacctgcagactggctctggtgtactgcagcctgatcaaggctagggccagagagctgtcctccggacagaaggatcagggacaggctgctaacatgctgtgcgtggtggtgaatgacatggagcagctgaggctggtgatcggaaagctgccagctcagctggcttgggaagctctggagcagagggtgggggctgtgctggaacagggacagctgcagaacaccctgcacgctcagctgcagagcgctctggctggcctgggacacgagatcaggaccggcgtgagaaccctggctgaacagctggaagtgggcatcgccaagcacatccagaagctggtgggcgtgagggaaagcgtgctgccagaggatgccatcctgcccctgatgaagttcctggaagtggagctgtgctacatgaacaccaatctggtgcaggaaaatttctccagcctgctgaccctgctgtggacccacaccctgaccgtgctggtggaggctgctgctagccagaggtccagctccctggcttccaacagactgaagatcgccctgcagaatctggaaatctgcttccacgctgagggatgcggcctgccacccaaggccctgcacaccgctaccttccaggccctgcagagggacctggaactgcaggctgccagctccagggagctgatcagaaagtacttctgcagcagaatccagcagcaggccgaaaccacctccgaagagctgggcgctgtgaccgtgaaggctagctacagggcctccgaacagaagctgagagtggagctgctgagcgctagctccctgctgccactggactccaacggcagctccgatcccttcgtgcagctgaccctggaaccaaggcacgaattccccgagctggctgccagagaaacccagaagcacaagaaggacctgcaccccctgttcgatgaaaccttcgagttcctggtgcccgcagaaccctgcagaaaggctggggcttgcctgctgctgaccgtgctggactacgataccctgggcgctgacgatctggaaggcgaggccttcctgcccctgagagaagtgccaggcctgagcggatccgaagaacccggggaggtgccacagaccagactgccactgacctacccagcccccaatggcgatcccatcctgcagctgctggaaggcaggaagggcgacagagaggctcaggtgttcgtcagactcaggagacatagagcaaaacaggcatcacagcacgccctcagaccagccccctag

SEQ ID NO: 2 - DNA, 1815 bp, Artificial sequence
  Synthetic construct
atggcaccaagcgggctcaaggcagtcgtgggagaaaaaatcctcagcggagtcatcagaagtgtgaagaaagacggggagtggaaagtgctgatcatggaccaccccagcatgagaatcctgagctcctgctgcaagatgtccgacatcctggctgaaggcatcaccatcgtggaggacatcaataagaggagagaacccatcccaagcctggaggccatctacctgctgtccccaaccgagaaggctcaggctcagagagtgatccacctgccacagagcgtgcaggctctgatcaaggatttccagggcacccccaccttcacctacaaggccgctcacatcttcttcaccgacacctgccccgaaccactgttcagcgagctgggcaggtccagactggccaaggtggtgaagaccctgaaggaaatccacctggctttcctgccatacgaggcccaggtgttcagcctggatgctccccactccacctacaatctgtactgcccattcagggccgaggaaaggaccagacagctggaagtgctggcccagcagatcgctaccctgtgcgccaccctgcaggaataccccgccatcaggtacagaaagggcccagaggataccgctcagctggctcacgctgtgctggctaagctgaacgccttcaaggctgacaccccaagcctgggggagggaccagaaaagaccaggtcccagctgctgatcatggatagggctgctgaccccgtgagcccactgctgcacgagctgaccttccaggctatggcctacgatctgctggacatcgaacaggacacctacagatacgaaaccaccggcctgtccgaggctagagaaaaggccgtgctgctggatgaagacgatgacctgtgggtggagctgaggcacatgcacatcgctgacgtgagcaagaaggtgaccgaactgctgaggaccttctgcgagtccaagagactgaccaccgataaggccaatatcaaggacctgagccagatcctgaagaagatgccccagtaccagaaggagctgaacaagtactccacccacctgcacctggctgatgactgcatgaagcacttcaagggcagcgtggaaaagctgtgctccgtggagcaggatctggctatgggcagcgacgccgagggcgaaaagatcaaggattccatgaagctgatcgtgccagtgctgctggatgctgctgtgccagcttacgacaagatcagggtgctgctgctgtacatcctgctgagaaacggcgtgagcgaggaaaatctggccaagctgatccagcacgctaacgtgcaggcccacagctccctgatcaggaatctggagcagctgggcggcaccgtgaccaatccaggcggcagcggaaccagctccaggctggagcccagggaaagaatggagccaacctaccagctgtccagatggacccccgtgatcaaggatgtgatggaagacgccgtggaggataggctggacagaaatctgtggcccttcgtgagcgaccccgctccaaccgctagctcccaggctgctgtgtccgctagattcggccactggcacaagaacaaggctggaatcgaggctagggctggaccaagactgatcgtgtacgtgatgggcggcgtggctatgagcgaaatgagggccgcttacgaggtgaccagagccaccgagggcaagtgggaggtgctgatcggcagctcccacatcctgaccccaaccagatttctggacgacctcaaagccctggacaaaaaactggaagacatcgctctgccctaa

SEQ ID NO: 3 - protein, 1090 aa, Artificial sequence
  Synthetic construct
MATLLSHPQQRPPFLRQAIKIRRRRVRDLQDPPPQMAPEIQPPSHHFSPEQRALLYEDALYTVLHRLGHPEPNHVTEASELLRYLQEAFHVEPEEHQQTLQRVRELEKPIFCLKATVKQAKGILGKDVSGFSDPYCLLGIEQGVGVPGGSPGSRHRQKAVVRHTIPEEETHRTQVITQTLNPVWDETFILEFEDITNASFHLDMWDLDTVESVRQKLGELTDLHGLRRIFKEARKDKGQDDFLGNVVLRLQDLRCREDQWYPLEPRTETYPDRGQCHLQFQLIHKRRATSASRSQPSYTVHLHLLQQLVSHEVTQHEAGSTSWDGSLSPQAATVLFLHATQKDLSDFHQSMAQWLAYSRLYQSLEFPSSCLLHPITSIEYQWIQGRLKAEQQEELAASFSSLLTYGLSLIRRFRSVFPLSVSDSPARLQSLLRVLVQMCKMKAFGELCPNTAPLPQLVTEALQTGTTEWFHLKQQHHQPMVQGIPEAGKALLGLVQDVIGDLHQCQRTWDKIFHNTLKIHLFSMAFRELQWLVAKRVQDHTTVVGDVVSPEMGESLFQLYISLKELCQLRMSSSERDGVLALDNFHRWFQPAIPSWLQKTYNEALARVQRAVQMDELVPLGELTKHSTSAVDLSTCFAQISHTARQLDWPDPEEAFMITVKFVEDTCRLALVYCSLIKARARELSSGQKDQGQAANMLCVVVNDMEQLRLVIGKLPAQLAWEALEQRVGAVLEQGQLQNTLHAQLQSALAGLGHEIRTGVRTLAEQLEVGIAKHIQKLVGVRESVLPEDAILPLMKFLEVELCYMNTNLVQENFSSLLTLLWTHTLTVLVEAAASQRSSSLASNRLKIALQNLEICFHAEGCGLPPKALHTATFQALQRDLELQAASSRELIRKYFCSRIQQQAETTSEELGAVTVKASYRASEQKLRVELLSASSLLPLDSNGSSDPFVQLTLEPRHEFPELAARETQKHKKDLHPLFDETFEFLVPAEPCRKAGACLLLTVLDYDTLGADDLEGEAFLPLREVPGLSGSEEPGEVPQTRLPLTYPAPNGDPILQLLEGRKGDREAQVFVRLRRHRAKQASQHALRPAP

SEQ ID NO: 4 - protein, 604 aa, Artificial sequence
  Synthetic construct
MAPSGLKAVVGEKILSGVIRSVKKDGEWKVLIMDHPSMRILSSCCKMSDILAEGITIVEDINKRREPIPSLEAIYLLSPTEKAQAQRVIHLPQSVQALIKDFQGTPTFTYKAAHIFFTDTCPEPLFSELGRSRLAKVVKTLKEIHLAFLPYEAQVFSLDAPHSTYNLYCPFRAEERTRQLEVLAQQIATLCATLQEYPAIRYRKGPEDTAQLAHAVLAKLNAFKADTPSLGEGPEKTRSQLLIMDRAADPVSPLLHELTFQAMAYDLLDIEQDTYRYETTGLSEAREKAVLLDEDDDLWVELRHMHIADVSKKVTELLRTFCESKRLTTDKANIKDLSQILKKMPQYQKELNKYSTHLHLADDCMKHFKGSVEKLCSVEQDLAMGSDAEGEKIKDSMKLIVPVLLDAAVPAYDKIRVLLLYILLRNGVSEENLAKLIQHANVQAHSSLIRNLEQLGGTVTNPGGSGTSSRLEPRERMEPTYQLSRWTPVIKDVMEDAVEDRLDRNLWPFVSDPAPTASSQAAVSARFGHWHKNKAGIEARAGPRLIVYVMGGVAMSEMRAAYEVTRATEGKWEVLIGSSHILTPTRFLDDLKALDKKLEDIALP

SEQ ID NO: 5 - DNA, 3273 bp, Artificial sequence
  Synthetic construct
atggcgacactcctctcccatccgcagcagcgccctcccttcttgcgccaggccatcaagataaggcgccgcagagtcagagatctacaggatcccccgccccaaatggccccggagatccagcctccatcccaccacttctcccccgagcagcgggccctgctctacgaggacgcactctacactgtcttgcaccgcctgggtcatcctgagcccaaccatgtgacggaggcctctgagctgctgcgatacctgcaggaggccttccacgtggagcccgaggagcaccagcagacactgcagcgggtcagggagcttgagaagccaatattttgtctgaaggcaacagtgaaacaggccaagggcattctgggcaaagatgtcagtgggttcagcgacccctactgcctgctgggcattgagcagggggtaggtgtgccagggggcagccccgggtcccggcatcggcagaaggctgtggtgaggcacaccatccccgaggaggagacccaccgcacgcaggtcatcacccagacactcaaccccgtctgggacgagaccttcatcctggagtttgaggacatcaccaatgcgagctttcatctggacatgtgggacctggacactgtggagtctgtccgacagaagcttggggagctcacggatctgcatgggcttcgcaggatctttaaagaggcccggaaggacaaaggccaggacgactttctggggaacgtggttctgaggctgcaggacctgcgctgccgagaggaccagtggtaccccctggaaccccgcactgagacctacccagaccgaggccagtgccacctccagttccaactcatccataagcggagagccacttcggccagccgctcgcagccgagctacaccgtgcacctccacctcctgcagcagcttgtgtcccacgaggtcacccagcacgaggcgggaagcacctcctgggacgggtcgctgagtccccaggctgccaccgtcctctttctgcacgccacacagaaggacctatccgacttccaccagtccatggcgcagtggctggcctacagccgcctctaccagagcctggagttccccagcagctgcctcctgcaccccatcaccagcatcgagtaccagtggatccagggtcggctcaaggcagaacagcaggaggagctggccgcctcattcagctccctgctgacctacggcctctccctcatccggaggttccgctctgtcttccccctctctgtctcggactccccagcccggctgcagtctcttctcagggtcctggtacagatgtgcaagatgaaggcctttggagaactgtgccccaacaccgccccattgccccagctggtgactgaggccctgcagactggcaccactgaatggttccacctgaagcagcagcaccatcaacccatggtgcagggcatcccggaggcaggcaaggccttgctgggcctggtacaggatgtcattggcgacctgcaccagtgccagcgcacatgggacaagatcttccacaataccctcaagatccacctcttctccatggctttccgggagctgcagtggctggtggccaagcgggtgcaggaccacacgacggttgtgggtgatgtagtgtccccagagatgggcgagagtctgttccagctctacatcagcctcaaggagctctgccagctgcgcatgagctcctcagagagggatggagtcctggccctggataatttccaccgctggttccagccggccatcccctcctggctgcagaagacgtacaacgaggccctggcgcgggtgcagcgcgctgtgcagatggatgagctggtgcccctgggtgaactgaccaagcacagcacatcagcggtggatctatccacctgctttgcccagatcagccacactgcccggcagctggactggccagacccagaggaggccttcatgattaccgtcaagtttgtggaggacacctgtcgcctggccctggtgtactgcagccttataaaggcccgggcccgcgagctctcttcaggccagaaggaccaaggccaggcagccaacatgctgtgtgtggtggtgaatgacatggagcagctgcggctggtgatcggcaagttgcccgcccagctggcatgggaggccctggagcagcgggtaggggccgtgctggagcaggggcagctgcagaacacgctgcatgcccagctgcagagcgcgctggccgggctgggccatgagatccgcactggcgtccgcaccctggccgagcagttggaggtgggcatcgccaagcacatccagaaactggtgggcgtcagggagtctgtcctgcctgaggatgccattctgcccctgatgaagttcctggaggtggagctttgctacatgaacaccaacttggtgcaggagaacttcagcagcctcctgaccctgctctggacccacacactcacagtgctggtggaggcggccgcctcccagcgcagctcatccctggcttccaacaggctgaagattgccctgcagaacctggagatctgcttccacgctgagggctgtggcctgccacccaaggccctgcacactgccaccttccaggctctgcagagggacctggagctgcaggcggcctccagccgggaactcatccggaagtacttctgcagccgaatccagcagcaggcagaaaccacctctgaggagctgggggctgtgacagtcaaggcctcctaccgcgcctctgagcagaagctgcgtgtggagctgctcagcgcctccagcctgctgcccctggactccaatggctccagcgacccctttgtccagctgaccttggagcccaggcatgagttccctgagctggccgcccgggagacccagaagcacaagaaggaccttcacccattgtttgatgagacctttgaattcctggtgcctgctgagccgtgccgcaaggctggggcatgcctcctgctcaccgtgctggactacgacacgctgggggccgacgacctggaaggcgaggccttcctgccgctgcgtgaggtgcccgggctgagtggctctgaggagcctggtgaggtgcctcagacccgcctgcccctcacgtaccccgcacccaacggggacccaatcctgcagctgctggagggccggaagggtgaccgagaagcccaggtctttgtgaggctgcggcggcaccgggccaagcaggcctcccagcatgccttgcggccggcaccgtag

SEQ ID NO: 6 - protein, 1090 aa, Artificial sequence; identical to SEQ ID NO: 3
  Synthetic construct

SEQ ID NO: 7 - DNA, 1815 bp, Artificial sequence
  Synthetic construct
atggcgccctcggggctgaaggcggtggtgggggaaaaaattctgagcggagttattcggagtgtcaagaaggatggggagtggaaggtgcttatcatggatcacccaagcatgcgcatcttgtcttcctgctgcaaaatgtcagatatcctggctgagggcatcaccattgttgaagacatcaacaaacggcgggaacccattcccagtctggaggccatttatttgctgagccccacggagaaggctcaggcccagagagtgatccaccttccccagtcggttcaggccctgatcaaagacttccaggggaccccgactttcacctacaaagcggcccatatcttcttcaccgacacctgccccgagcccctgttcagtgagctaggccgctctcgtctggcaaaggtggtgaagacgttgaaggagattcaccttgccttcctcccctacgaggcccaggtgttctccctcgatgctccccacagcacctacaacctctactgccccttccgggcagaggagcgcacgcggcagctcgaggtgctggcccagcagattgccacgctgtgcgccaccctgcaggagtacccggccatccgctaccgcaagggcccagaggacacagcccagttggcccacgccgtcctggccaagctgaacgccttcaaggcagacactcccagtctgggcgagggcccagagaaaacccgctcccagctgctgataatggaccgggcagctgaccccgtgtccccactactgcatgagctcacgttccaggccatggcgtatgatctgctggacatagagcaggacacatacaggtatgagaccaccgggctgagcgaggcgcgggagaaggccgtcttgctggacgaggacgatgacttgtgggtggagcttcgccacatgcatatcgcagatgtgtccaagaaggtcacggagctcctgaggaccttctgtgagagcaagaggctgaccacggacaaggcgaacatcaaagacctatcccagatcctgaaaaagatgccgcagtaccagaaggagctgaataagtattctacgcacctgcatctagcagatgattgtatgaagcacttcaagggctcggtggagaagctgtgtagtgtggagcaggacctggccatgggctccgacgcagagggggagaagatcaaggactccatgaagctgatcgttccggtgctgctggacgcggcggtgcccgcctacgacaagatccgggtcctgctgctctacatcctccttcggaatggtgtgagtgaggagaacctggccaagctgatccagcatgccaatgtacaggcgcacagcagcctcatccgtaacctggagcagctgggaggcactgtcaccaaccccgggggctcggggacctccagccggctggagccgagagaacgcatggagcccacctatcagctgtcccgctggaccccggtcatcaaggatgtaatggaggacgccgtggaggaccggctggacaggaacctgtggcccttcgtatccgaccccgcccccacggccagctcccaggccgctgtcagtgcccgcttcggtcactggcacaagaacaaggctggcatagaagcccgggcgggcccccggctcatcgtgtatgtcatgggcggtgtggccatgtcagagatgagggccgcctacgaggtgaccagggccaccgagggcaagtgggaggtgctcattggctcctcacacatcctcaccccgacccgcttcctggatgacctgaaggcactggacaagaagctggaggacattgccctgccctga

SEQ ID NO: 8 - protein, 604 aa, Artificial sequence; identical to SEQ ID NO: 4
  Synthetic construct

SEQ ID NO: 9 - DNA, 24 bp, Artificial sequence
  Synthetic construct
tggagtgggacagagaaattaaca

SEQ ID NO: 10 - DNA, 21 bp, Artificial sequence
  Synthetic construct
gctggttttgcgattcttcaa

SEQ ID NO: 11 - DNA, 13 bp, Artificial sequence
  Synthetic construct
gccgccrccaugg

SEQ ID NO: 12 - DNA, 11 bp, Artificial sequence
  Synthetic construct
caccatggcgg

SEQ ID NO: 13 - DNA, 11 bp, Artificial sequence
  Synthetic construct
cgccatggcgg

SEQ ID NO: 14 - DNA, 11 bp, Artificial sequence
  Synthetic construct
cacgatggcgg

SEQ ID NO: 15 - DNA, 11 bp, Artificial sequence
  Synthetic construct
caccatgacgg

SEQ ID NO: 16 - DNA, 11 bp, Artificial sequence
  Synthetic construct
cgcgatggcgg

SEQ ID NO: 17 - DNA, 11 bp, Artificial sequence
  Synthetic construct
cgccatgacgg

SEQ ID NO: 18 - DNA, 11 bp, Artificial sequence
  Synthetic construct
cacgatgacgg

SEQ ID NO: 19 - DNA, 11 bp, Artificial sequence
  Synthetic construct
cgcgatgacgg

SEQ ID NO: 20 - DNA, 10 bp, Artificial sequence
  Synthetic construct
gccaccatgg

SEQ ID NO: 21 - DNA, 1187 bp, Artificial sequence
  Synthetic construct
atcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

(19) United States
(12) Patent Application Publication
(10) Pub. No.: US 2021/0393687 A1
(43) Pub. Date: Dec. 23, 2021

(54) COMPOSITIONS AND METHODS FOR TREATMENT OF HEMOPHAGOCYTIC LYMPHOHISTIOCYTOSIS

(71) Applicants: EMORY UNIVERSITY, Atlanta, GA (US); CHILDREN'S HEALTHCARE OF ATLANTA, INC., Atlanta, GA (US)

(72) Inventors: Harold Trent Spencer, Atlanta, GA (US); Christopher Doering, Atlanta, GA (US); Shanmuganathan Chandrakasan, Atlanta, GA (US); Sarah Takushi, Atlanta, GA (US)

(73) Assignees: EMORY UNIVERSITY, Atlanta, GA (US); CHILDREN'S HEALTHCARE OF ATLANTA, INC., Atlanta, GA (US)

(21) Appl. No.: 17/281,738

(22) PCT Filed: Oct. 1, 2019
(86) PCT No.: PCT/US2019/054125

§ 371 Date: Mar. 31, 2021

Related U.S. Application Data
(60) Provisional application No. 62/739,593, filed on Oct. 1, 2018.

(51) Int. Cl. A61K 35/17; C07K 14/47; C07K 14/705; C12N 15/86; C12N 5/0783; A61P 37/06; A61K 48/00

(52) U.S. Cl. CPC A61K 35/17; C07K 14/47; C07K 14/705; C12N 15/86; C12N 5/0636; C12N 2740/15071; A61K 48/0066; C12N 2506/1353; C12N 2510/00; C12N 2740/15043; A61P 37/06

(57) ABSTRACT

Provided herein are nucleic acids, vectors, and cells containing an expression-optimized codon that encodes a Munc13-4 polypeptide or a STXBP2 polypeptide. Also provided are methods of making and using the nucleic acids, vectors, and cells. Also provided herein are methods of treating of Hemophagocytic Lymphohistiocytosis (HLH) in a subject.

This application claims the benefit of U.S. Provisional Application No. 62/739,593, filed Oct. 1, 2018, which is hereby incorporated herein in its entirety by this reference.

### BACKGROUND

Hemophagocytic Lymphohistiocytosis (HLH) is a syndrome marked by impairment or absence of cytotoxic function by NK and CD8+ T cells with activation of the immune system. The impaired cytotoxic function present in HLH leads to hypercytokinemia and hemophagocytosis. These in turn cause all the typical symptoms of HLH, for example, prolonged fever, splenomegaly, hepatomegaly, cytopenia, hyperferritinemia, hypertriglyceridemia, hypofibrinogenemia, hemophagocytosis, hypercytokinemia, and/or lymphohistiocytic infiltrate, bone marrow hypoplasia, meningeal infiltrate. Among the cytokines elevated in HLH patients are IFNγ, interleukin 6 (IL-6), IL-10, tumor necrosis factor (TNF) a, IL-8, macrophage colony stimulating factor (MCSF) and granulocyte-macrophage colony-stimulating factor (GM-CSF).

HLH includes primary (genetic/familial HLH (FLH)) and secondary HLH, both clinically manifested by a dysregulation of the immune system leading to a profound hypercytokinemia with deleterious consequences on various tissues and organs. Familial HLH-3 (FLH-3) is classified by a genetic mutation in the gene UNC13D affecting the function or expression of the Munc13-4 protein, which participates in vesicle priming. More specifically, Munc13-4 is essential for the exocytosis of perforin- and granzyme-containing granules from cytotoxic cells. Without this function, cells are able to recognize an immunological insult and release inflammatory cytokines but are unable to execute their cytotoxic functions. The result is a hyper-inflammatory state that, if left untreated, is fatal.

Familial HLH-5(FLH-5) is associated with a genetic mutation in the gene STXBP2 (UNC18B) affecting the function or expression of Munc18-2 protein, which participates in vesicle transport and fusion. Primary HLH is typically a heterogeneous autosomal recessive disorder with symptoms generally first seen in infancy and early childhood. When the infant or child encounters an immunological insult such as a viral infection (e.g., Epstein Barr Virus), their lymphocytes cannot execute cytotoxic degranulation, leading to a positive feedback loop of inflammation. If untreated the disease is typically fatal, and a significant portion of patients die despite current treatment options. To date, the only curative treatment available for HLH patients is an allogenic bone marrow transplant. However, inflammation must be adequately controlled prior to transplant, and only 53% of patients respond fully to pre-transplant conditioning. For patients that receive a transplant, the 5-year survival rate is 54%±6%, though results vary depending on the conditioning protocol. Thus, there is a need to identify improved methods for managing HLH.

### SUMMARY

Provided herein is a nucleic acid sequence comprising an expression-optimized nucleic acid sequence encoding a Munc13-4 polypeptide. The expression-optimized nucleic acid sequence encoding a Munc13-4 polypeptide optionally comprises the nucleic acid sequence of SEQ ID NO: 1 or a sequence having at least 95% identity to SEQ ID NO: 1. Also provided are polypeptides encoded by the nucleic acid sequence comprising the expression-optimized nucleic acid sequence encoding a Munc13-4 polypeptide, such as a sequence comprising SEQ ID NO:3 or an amino acid sequence having at least 95% sequence identity with SEQ ID NO:3. Further provided are expression vectors comprising the nucleic acid sequence that includes the expression-optimized nucleic acid sequence encoding a Munc13-4 polypeptide and cells comprising the expressing vector or the nucleic acid sequence that includes an expression-optimized nucleic acid sequence encoding a Munc13-4 polypeptide. Optionally, the vector also includes a promoter.

Also provided herein is a nucleic acid sequence comprising an expression-optimized nucleic acid sequence encoding a STXBP2 polypeptide. The expression-optimized nucleic acid sequence encoding a STXBP2 polypeptide comprises SEQ ID NO: 2 or a sequence having at least 95% identity to SEQ ID NO: 2. Also provided are polypeptides encoded by the nucleic acid sequence comprising the expression-optimized nucleic acid sequence encoding a STXBP2 polypeptide, such as a sequence comprising SEQ ID NO:4 or an amino acid sequence having at least 95% sequence identity with SEQ ID NO:4. Further provided are expression vectors comprising the nucleic acid sequence that includes the expression-optimized nucleic acid sequence encoding a STXBP2 polypeptide and cells comprising the expressing vector or the nucleic acid sequence that includes the expression-optimized nucleic acid sequence encoding a STXBP2 polypeptide. Optionally, the vector also includes a promoter.

In the cells described herein comprising a nucleic acid sequence or vector that includes the expression-optimized nucleic acid sequence encoding a Munc13-4 or STXBP2 polypeptide. The nucleic acid sequence optionally is stably integrated into the genome of the cell. The cell can be a hematopoietic stem cell or a hematopoietic stem cell lineage cell (e.g., a T cell).

Also provided herein is a method of making the cell described herein. The method includes introducing into the cell a nucleic acid sequence or vector that includes an expression-optimized nucleic acid sequence encoding a Munc13-4 or STXBP2 polypeptide.

Further provided are methods of treating familial hemophagocytic lymphohistiocytosis (HLH) in a subject by introducing into a population of cells obtained from the subject a nucleic acid or vector as described herein to provide a population of genetically modified cells expressing Munc13-4 or STXBP2 polypeptide; and transplanting the genetically modified cells into the subject. The method optionally further comprises culturing the genetically modified cells prior to transplantation into the subject. Culture conditions can promote expansion and/or differentiation depending on the cell type. The cells obtained from the subject can be, for example, hematopoietic stem cells or hematopoietic stem cell lineage cells, for example, T cells. Genetically modified hematopoietic stems cells can also be differentiated, for example, into T cells.

### DESCRIPTION OF THE FIGURES

FIG. 1A shows a diagram of a lentiviral construct as described herein.

FIG. 1B is Western blot confirming the expression of Munc13-4 in a healthy donor cells (Lane 1) and UNC13D+TPO-GFP transduced patient cells (Lane 2) but not non-transduced patient cells (Lane 3).

FIG. 1C shows flow cytometry results indicating that stimulation of transduced T cells using CD3 and CD28 antibody for four hours induces higher surface expression of CD107a (Lamp-1) as compared to non-transduced cells.

FIG. 1D shows the flow cytometry results of a cell killing assay performed by culturing transduced T cells with MPL-expressing target cells. Cell killing ability was compared between non-transduced and transduced patient cells. Killed cells were identified as being positive for phosphatidyl inositol (PI) and Annexin V.

FIG. 2A shows diagrams of modified lentiviral vector constructs.

FIG. 2B is a bar graph showing that modification to the original lentiviral construct did not produce any change in viral titer as assessed by transduction of HEK 293T cells (Kruskal-Wallis H test, p=0.48).

FIG. 2C shows expression data in a Western blot indicating that a codon-optimized version of the UNC13D gene (under the control of the Ef1α promoter and including a Kozak) was expressed more highly in NIH 3T3 cells than the non-codon-optimized version of the gene.

FIG. 2D is a Western blot showing that transduction of NIH 3T3 cells with the expression-optimized-UNC13D construct containing an Ef1α promoter and Kozak sequence showed high levels of transgene expression, beyond normal physiological levels observed in the peripheral blood mononuclear cells (PBMCs) of healthy humans.

FIG. 3A is a bar graph showing results of flow cytometry analysis from mice transplanted six seeks earlier with mixtures of Jinx and wildtype bone marrow. The results show that chimerism was maintained within the bulk peripheral blood and also across the CD8+, CD4+, and NK1.1+ cell populations.

FIG. 3B shows levels of CD69 and CD107a expression before (gray) and after (black) stimulation with CD3 and CD28 antibodies in CD8+CD44+ splenocytes from non-transplanted C57BL/6 mice (WT) and in CD45.1+ engrafted cells from chimerically transplanted Jinx mice. (Mann Whitney test, p=0.2253 for CD69 expression, p=0.8846 for CD107a expression.)

FIG. 3C shows the activation ability of CD44+CD8+ splenocytes as compared to CD44+CD8+ splenocytes from LCMV-infected WT and Jinx mice. Thirty weeks after transplant, the chimerically transplanted mice were infected intraperitoneally with 2×10⁵ PFU LCMV Armstrong to re-capitulate the FHL3 disease model. Eight days after infection, the mice were sacrificed, and their CD44+CD8+ splenocytes were assessed for the ability to activate (right, Spearman's rank-order correlation, two tailed: r=0.2105, p=0.508) and degranulate (left, Spearman's rank-order correlation, two tailed: r=0.814, p=0.002) as compared to the CD44+CD8+ splenocytes from LCMV-infected WT and Jinx mice.

FIG. 4A shows a diagram of an experimental design.

FIG. 4B shows complete blood count (CBC) data in mice twelve weeks post-transplant. WBC, leukocyte, and lymphocyte populations showed no difference between gene therapy modified mice and mice that were transplanted with CD45.1 Sca-1 cells (unpaired, two-tailed t-test, p=0.82, 0.84, 0.79 and 0.42 for WBCs, leukocytes, lymphocytes, and platelets respectively).

FIG. 4C shows flow cytometry when splenocytes were gated using Live/Dead stain and CD8, and CD44 cell surface markers were used to identify the memory CD8 T cells, which were analyzed in subsequent degranulation studies.

FIG. 4D shows flow cytometry results detecting up-regulation of cell surface CD107a with and without stimulation with CD3/CD28 antibody in wildtype, Jinx and mice treated with CD45.1 Sca-1 cells.

FIG. 4E is a bar graph showing the percent change in CD107a expression in gene therapy-modified mice as compared to wildtype and Jinx mice. Gene-therapy modified mice showed significantly greater upregulation (two-tailed Mann Whitney U test, p=0.005).

FIG. 5A shows flow cytometry results for CD44 in LCMV Armstrong infected in naïve C57BL/6 mice, mice with a primary infection, and mice with a secondary infection. Consistent with previous descriptions of the FHL3 mouse model induces a robust immunological response.

FIG. 5B shows the percentage of CD8+/CD44+ cells in naïve C57BL/6 mice, mice with a primary infection, and mice with a secondary infection.

FIG. 5C shows the percentage of CD8+/CD44+ cells in wildtype and Jinx mice.

### DETAILED DESCRIPTION

Described herein are compositions and methods related to an expression-optimized nucleic acid sequence encoding either a Munc13-4 polypeptide or a STXBP2 polypeptide. As used herein, Munc13-4 polypeptide and STXBP2 polypeptide refer to wildtype or variants of wildtype Munc13-4 and STXBP2 that retain one or more cellular functions of the wildtype Munc13-4 and STXBP2, respectively. The compositions and methods described herein are useful for treating HLH in a subject, a disorder in which Munc13-4 polypeptide or STXBP2 polypeptide are either not expressed or are expressed in a mutated form that reduces the cellular function of the polypeptide.

Munc13-4 is also referred to as UNC-13 homolog D protein, HPLH3, FHL3, and HLH3. The wildtype nucleic acid sequence for human Munc13-4 is provided as SEQ ID NO:5 and the wildtype amino acid sequence is provided as SEQ ID NO:6. An exemplary expression-optimized nucleic acid sequence encoding a Munc13-4 polypeptide is provided as SEQ ID NO:1. Also provided herein are expression-optimized nucleic acid sequences having at least 75%, 80%, 85%, 90% or 95% identity to SEQ ID NO: 1.

STXBP2 is also known as Syntaxin-Binding Protein 2, Unc-18 homolog B protein, and Munc18-2. The wildtype nucleic acid sequence for human STXBP2 is provided as SEQ ID NO:7 and the wildtype amino acid sequence is provided as SEQ ID NO:8. An exemplary expression-optimized nucleic acid sequence encoding a STXBP2 polypeptide is provided as SEQ ID NO:2. Also provided herein are expression-optimized nucleic acid sequences having at least 75%, 80%, 85%, 90% or 95% identity to SEQ ID NO: 2.

As used herein expression-optimized refers to a codon optimized for increased expression, as compared to expression of a wildtype codon. Thus, an expression-optimized nucleic acid sequence encoding either a Munc13-4 or a STXBP2 polypeptide provides higher expression than a wildtype (non-mutated) nucleic acid sequence encoding a Munc13-4 or STXBP2 polypeptide. The difference in expression is at least about 15% greater, at least 20% greater, or at least 25% greater.

Also provided herein are polypeptides encoded by the nucleic acid sequences described herein. For example, the Munc13-4 polypeptide sequence comprises the amino acid sequence of SEQ ID NO:3. For example, the STXBP2 polypeptide sequences comprises amino acid sequence of SEQ ID NO:4. Further provided are amino acid sequences having at least 75%, 80%, 85%, or 95% identity as compared to SEQ ID NO:3 or SEQ ID NO:4, wherein the amino acid sequence is not a wildtype Munc13-4 or STXBP2 polypeptide sequence. Further provided are amino acid sequences having at least 75%, 80%, 85%, or 95% identity as compared to SEQ ID NO:3 or SEQ ID NO:4, wherein the amino acid sequence is not SEQ ID NO: 6 or SEQ ID NO: 8.

Further provided are expression vectors comprising the nucleic acid sequence that includes the expression-optimized nucleic acid sequence encoding a Munc13-4 polypeptide or STXBP2 polypeptide. The vector can be a viral vector (e.g., a lentiviral vector, a retroviral vector, an adenoviral vector, an adeno-associated viral vector, etc.) or a non-viral vector (e.g., a plasmid, transposase vectors, etc.). Optionally, the vector includes one or more transcriptional regulatory non-coding sequences. The expression vector optionally comprises a promoter operably linked to the nucleic acid encoding either a Munc13-4 or a STXBP2 polypeptide. Examples of promoters include exogenous promoters such as an Ef1α promoter. Other promoters that can be used include, but are not limited to, a human ubiquitin C (UBC) promoter, a murine leukemia virus-derived MND promoter (myeloproliferative sarcoma virus enhancer, negative control region deleted, d1587rev primer-binding site substituted), a murine stem cell virus promoter (MSCV LTR), or a cytomegalovirus (CMV) promoter. See, for example, Norman et al. “Quantitative Comparison of Constitutive Promoters in Human ES Cells,” PLoS ONE 5(8): e12413. Optionally the vector further comprise a Kozak consensus sequence. A Kozak consensus sequence occurs in eukaryotic mRNA and has the consensus sequence (gcc)gccRccAUGG (SEQ ID NO: 11). A lower case letter denotes the most common base at a position where the base can nevertheless vary. Upper case letters in the consensus sequence indicate highly conserved bases, i.e., the AUGG sequence is constant or rarely, if ever, changes, with the exception being the IUPAC ambiguity code “R” which indicates that a purine (adenine or guanine) is always observed at this position. The sequence in parentheses (gcc) is of uncertain significance. Examples of Kozak consensus sequences include, but are not limited to, CACCATGGCGG (SEQ ID NO: 12), CGCCATGGCGG (SEQ ID NO: 13), CACGATGGCGG (SEQ ID NO: 14). CACCATGACGG (SEQ ID NO: 15), CGCGATGGCGG (SEQ ID NO: 16), CGCCATGACGG (SEQ ID NO: 17), CACGATGACGG (SEQ ID NO: 18) and CGCGATGACGG (SEQ ID NO: 19). Optionally, the Kozak sequence is GCCACCATGG (SEQ ID NO: 20). Optionally the promoter sequence comprises or consists of SEQ ID NO: 21, an exemplary Ef1α promoter. Optionally, the vector includes one or more one or more linker sequences that separate the components of the nucleic acid construct. The linker sequence can be two, three, four, five, six, seven, eight, nine, ten amino acids or greater in length.

Also provided herein is a cell comprising a nucleic acid sequence or a vector comprising the nucleic acid that includes the expression-optimized nucleic acid sequence encoding a Munc13-4 polypeptide or STXBP2 polypeptide. Optionally, the nucleic acid sequence is stably integrated into the genome of the cell.

The cell comprising a nucleic acid sequence or a vector comprising a nucleic acid sequence that includes the expression-optimized nucleic acid sequence encoding a Munc13-4 polypeptide or STXBP2 polypeptide is optionally a hematopoietic stem cell or a cell of hematopoietic stem cell lineage, such as a T cell. In some embodiments, the human cell is a hematopoietic cell, for example, an immune cell, such as a hematopoietic stem cell, a T cell, a B cell, a macrophage, a natural killer (NK) cell or dendritic cell.

The T cell comprising the expression-optimized nucleic acid can be a regulatory T cell, an effector T cell, a naïve T cell, a T helper cell, a cytotoxic T cell, a natural killer T cell, or a γδ T cell. In the methods and compositions provided herein, the human T cells can be primary T cells. The effector T cell can be a CD8+ T cell. The T cell can be a CD4+ cell. Optionally, the T cell is a CD4+CD8+ T cell or a CD4−CD8− T cell. Optionally, the T cell is a T cell that expresses a TCR receptor or differentiates into a T cell that expresses a TCR receptor. Optionally, the T cells is a CD3+ T cell. Optionally, the T cell is selected as a CD3+ cell. Populations of any of the cells modified by any of the methods described herein are also provided. The cell can be in vitro, ex vivo, or in vivo.

Human hematopoietic stem cells are typically selected for expression of CD34 and optionally CD90, C-kit/CD117, CD133, CD150 and the absence of CD38 and the absence of lineage-specific marker expression such as CD2, CD3, CD11b, CD11c, CD14, CD16, CD19, CD24, CD56, CD66b and CD235.

### Methods of Making

Provided herein is a method of making a cell or a population of cells comprising a nucleic acid or vector that includes the expression-optimized nucleic acid sequence encoding a Munc13-4 polypeptide or STXBP2 polypeptide. The expression-optimized nucleic acid can be introduced into the cell using any available method. For example, the nucleic acid sequence can be introduced into the cell using a viral or non-viral vector or by targeted nuclease-mediated insertion of the nucleic acid sequence into the cell. The targeted nuclease is selected from the group consisting of an RNA-guided nuclease (e.g., a Cas9 nuclease or a Cpf1 nuclease), a transcription activator-like effector nuclease (TALEN), a zinc finger nuclease (ZFN) or a megaTAL. The methods of making a cell or population of cells comprising a nucleic acid or vector provided herein can be performed in vitro, ex vivo or in vivo.

One or more nucleic acids can be introduced into a cell by transduction or transfection. The nucleic acids can be in the form of naked DNA or RNA, or the nucleic acids can be in a vector for delivering the nucleic acids to the cells. The vector can based on a lentiviral vector (as described in the Examples), or by commercially available vector systems, such as an adeno-associated viral vector, a retroviral vector or an adenovirus vector (Quantum Biotechnologies, Inc. (Laval, Quebec, Canada). Delivery of the nucleic acid or vector to cells can be via a variety of mechanisms. As one example, delivery can be via chemical methods (e.g., using a transfection agent such as polybrene (Hexadimethrine bromide) or via a liposome, using commercially available liposome preparations such as LIPOFECTIN, LIPOFECTAMINE (GIBCO-BPvL, Inc., Gaithersburg, Md.), SUPERFECT (Qiagen, Inc. Hilden, Germany), or TRANSFECTAM (Promega Biotec, Inc., Madison, Wis.), as well as other liposomes developed according to procedures standard in the art.

In addition, the disclosed nucleic acid or vector can be delivered by physical means such as electroporation or nucleoporation. Electroporation methods are available from Genetronics, Inc. (San Diego, Calif.) as well as by means of a SONOPORATION machine (ImaRx Pharmaceutical Corp., Tucson, Ariz.). Methods for nucleoporation can also be used. See, for example, Maasho et al. “Efficient gene transfer into the human natural killer cell line, NKL, using the amaxa nucleofection system,” Journal of Immunological Methods 284(1-2): 133-140 (2004); and Aluigi et al. “Nucleofection is an efficient non-viral transduction technique for human bone marrow derived mesenchymal stem cells,” Stem Cells 24(2): 454-461 (2006).

A CRISPR/Cas system can also be used to stably integrate a heterologous sequence into the genome of the cell. Engineered CRISPR/Cas systems contain two components: a guide RNA (gRNA, also referred to as single guide RNA (sgRNA)) and a CRISPR-associated endonuclease. The gRNA is a short synthetic RNA composed of a scaffold sequence necessary for binding with the CRISPR-associated endonuclease and a user-defined ˜20 nucleotide spacer that defines the genomic target to be modified. Thus, one can change the genomic target of the CRISPR-associated endonuclease by simply changing the target sequence present in the gRNA. Where homology-directed repair (HDR) of the genomic target is desired, such as when the aim is to correct a mutated genomic sequence, the system includes a target template sequence that provides the desired sequence to be introduced into the genome in place of the mutated genomic sequence. When non-homologous end joining (NHEJ) is the repair mechanism used to repair the break in the genomic DNA, no target template sequence is used and repair generally results in deletions or insertions at the site of repair. This mechanism is useful where the desired outcome from gene editing is to inactivate and/or impair the function of a genomic sequence, or restoring the reading frame of a gene disrupted by deletions or insertions.

An expression-optimized nucleic acid can be introduced into a population of cells using (1) a guide RNA (gRNA) comprising a first nucleotide sequence that hybridizes to a target DNA in the genome of a cell, wherein the target DNA is a nucleic acid encoding a Munc13-4 or STXBP2 polypeptide with one or more mutations, and a second nucleotide sequence that interacts with a site-directed nuclease; (2) a recombinant site-directed nuclease, wherein the site-directed nuclease comprises an RNA-binding portion that interacts with the second nucleotide sequence of the guide RNA and wherein the site-directed nuclease specifically binds and cleaves the target DNA to create a double stranded break; and (3) a single-stranded donor oligonucleotide (ssODN) that hybridizes to a genomic sequence flanking the double stranded break in the target DNA and integrates into the target DNA to correct the mutation in the Munc13-4 or STXBP2. Optionally, a double stranded donor oligonucleotide can be used. Optionally, the targeted nuclease is a nickase, for example a Cas9 nickase or a Cpf1 nickase that introduces single stranded breaks in genomic DNA. Optionally, the components of the CRISPR-Cas9 system can be delivered using nucleic acid or vector-based delivery. For example, the guide RNA, donor oligonucleotide and a nucleic acid encoding the targeted nuclease can be delivered to a cell using one or more vectors. Optionally, the one or more vectors can be non-viral vectors. See, for example, “Non-viral delivery systems for CRISPR/Cas9-based genome editing: Challenges and opportunities,” Biomaterials 171: 207-218 (2018)). Optionally, the components of the CRISPR-Cas9 system can be delivered using viral vectors, for example, adenoviral vectors, lentiviral vectors, and the like. See, for example, Xu et al., “Viral Delivery Systems for CRISPR,” Viruses 11(1): 28 (2019)).

Optionally, in any of the methods provided herein, at least 5%, 10%, 15% or 20% of the cells in a population of cells are modified to include an expression optimized nucleic acid sequence encoding Munc13-4 or STXBP2. A population of cells can be enriched for cells comprising an expression optimized nucleic acid sequence encoding Munc13-4 or STXBP2.

Also provided herein are methods of making a pharmaceutical composition comprising a population of cells with a nucleic acid or vector that includes the expression-optimized nucleic acid sequence encoding a Munc13-4 polypeptide or STXBP2 polypeptide. The method comprises isolating cells, inserting the expression-optimized nucleic into the cells, and adding a pharmaceutically acceptable carrier. Examples of the pharmaceutically-acceptable carriers include, but are not limited to, saline, Ringer's solution and dextrose solution. The pH of the solution is preferably from about 5 to about 8, and more preferably from about 7 to about 7.5. Carriers include sustained release preparations, e.g., films, liposomes, nanoparticles, or the like.

Cell can be isolated from blood or bone marrow of one or more subjects. For example, bone marrow cells can be obtained or harvested from a subject Bone marrow harvesting involves collecting stem cells with a needle placed into the soft center of the bone, the marrow. Bone marrow can be harvested for example, from the hip bones or sternum of the subject. From about 500 ml to about 1 liter of bone marrow can be obtained from the subject.

By way of example, when whole blood is centrifuged, cells are separated in density layers, optionally using an apheresis machine. Plasma is the top, and red blood cells on the bottom. A buffy coat, which contains platelets and white blood cells, sits in between the plasma and red blood cells. The white blood cells contain T cells. T cells or hematopoietic stem cells can be isolated from the buffy coat, mononuclear cells, or bone marrow using fluorescence-activated cell sorting (FACS). Alternatively, antibodies that bind cells markers bound to magnetic material can be used with a magnet to isolate (negatively or positively) the bound cells followed by use of a releasing agent to remove the cells from the beads. Cells can also be isolated directly from whole blood or bone marrow by FACS or immunomagnetic selection. Selection can be based on recognition of specific surface markers. Human hematopoietic stem cells are typical selected for expression of CD34 and optionally CD90, C-kit/CD117, CD133, CD150 and the absence of CD38 and the absence of lineage-specific marker expression such as CD2, CD3, CD11b, CD11c, CD14, CD16, CD19, CD24, CD56, CD66b, and CD235.

Optionally, the method further comprises culturing the cells. Culture conditions can be selected for expansion of the cells or for differentiation into a selected cell type. See, for example, Kita et al. “Ex vivo expansion of hematopoieitc stem and progenitor cells: Recent advances,” World Journal of Hematology 3(2): 18-28 (2014).

### Methods of Use

Mutations in Munc13-D and STXBP2 constitute the majority of known genetic causes of primary HLH patients. This disclosure contemplates a curative treatment in which cells are isolated from a subject, the cells are genetically engineered ex vivo using recombinant viral vectors or other methods of gene transfer, followed by administration of the genetically-engineered stem cell product to the subject.

Provided herein is a method of treating familial hemophagocytic lymphohistiocytosis (HLH) in a subject comprising introducing into a population of cells obtained from the subject the nucleic acid with the expression-optimized nucleic acid sequence encoding either a Munc13-4 polypeptide or a STXBP2 polypeptide or introducing into a population of cells the vector comprising the nucleic acid with the expression-optimized nucleic acid sequence encoding either a Munc13-4 polypeptide or a STXBP2 polypeptide. The population of genetically modified cells produced is then administered to the subject.

Optionally, prior to the administration step, the genetically modified cells are cultured under selected conditions. The conditions can be selected to promote expansion and/or differentiation as described above. The cells obtained from the subject are optionally hematopoietic stem cells or cells of hematopoietic cell lineage (e.g., T cells). Optionally, the cells administered to the subject are hematopoietic stem cells or T cells. The T cells can be T helper cells, cytotoxic T cells, natural killer T cells or γδ T cells. The isolated cells or administered cells are optionally NK cells.

Optionally the cells are administered to the same subject from which the cells (or their precursors) are isolated. First, modification of a patient's own bone marrow or cells would reconstitute a patient's immune system with corrected cytotoxic cells, correcting the HLH phenotype for many years post-transplant. Second, this autologous transplant would reduce the risk of graft vs. host disease compared to an allogenic transplant. Third, subjects that receive an HLH diagnosis would no longer have to wait to find a matching transplant donor. Finally, modification of a patient's own CD8+ cells could be used in autologous adoptive transfer to control inflammation prior to transplant.

Alternatively, the cells can be isolated from a first subject and then the cells or their progeny administered to a second subject. Optionally, cells are isolated from multiple subjects and pooled prior to administration and/or culturing.

Also provided is a method of treating familial hemophagocytic lymphohistiocytosis (HLH) in a subject comprising: (a) introducing into a population of pluripotent stem cells the nucleic acid of any one of claims 1-4 or the vector of any one of claims 6-9 to provide a population of genetically modified cells; (b) differentiating the genetically modified cells or progeny thereof into a population of differentiated cells that require Munc13-4 or STXB2 function; and (c) transplanting the population of differentiated cells of step (b) into the subject. Optionally, the method further comprises culturing the genetically modified cells prior to transplantation into the subject. Optionally, culturing comprises conditions for expansion. As used throughout a cell that requires Munc13-4 or STXB2 function is a cell that expresses Munc13-4 or STXB2. See, for example, Yamanaka “Strategies and New Developments in the Generation of Patient-Specific Pluripotent Stem Cells, Cell Stem Cell 1(1): 39-49 (2007); and Chang et al. “Polycistronic Lentiviral Vector for ‘Hit and ‘Run’’ Reprogramming of Adult Skin Fibroblasts to Induced Pluripotent Stem Cells, Stem Cells 27(5): 1042-1049 (2009)).

As used herein, transplanting, introducing or administering cells to a subject refers to the placement of cells into a subject. For example, the cells described herein comprising an expression-optimized nucleic acid according to the methods described herein can be transplanted into a subject by an appropriate route that results in at least partial localization of the transplanted cells at a desired site. The cells can be implanted directly to the desired site, or alternatively can be administered by any appropriate route which results in delivery to a desired location in the subject where at least a portion of the implanted cells remain viable. For example, the cells can be administered systemically via intravenous infusion. The period of viability of the cells after administration to a subject can be as short as a few hours, e. g. twenty-four hours, to a few days, to as long as several years.

An effective dose or amount of corrected cells is administered to the subject. The terms effective amount and effective dosage are used interchangeably. The term effective amount is defined as any amount necessary to produce a desired physiologic response. In some methods, about 1×10⁶ to about 7×10⁶ corrected cells/kg can be administered, but this amount can vary depending on a number of factors, including the percentage of corrected cells resulting from the method, severity of the disease, age and condition of the subject, and the like. Effective amounts and schedules for administering the cells may be determined empirically, and making such determinations is within the skill in the art. The dosage ranges for administration are those large enough to produce the desired effect (e.g., treatment of a disease, for example, HLH). The dosage should not be so large as to cause substantial adverse side effects, such as unwanted cross-reactions, anaphylactic reactions, and the like.

Further provided are in vivo methods of modifying a cell or a population of cells in a subject. For example, provided herein is a method of treating HLH in a subject comprising introducing into the subject an expression optimized nucleic acid sequence encoding a Munc13-4 or STXB2 polypeptide to provide a population of genetically modified cells in the subject. The in vivo methods described herein can be performed in combination with any of the ex vivo methods of modifying hematopoietic stem cell lineage cells, for example, T cells, described herein. In vivo modification of cells in a subject can be performed prior to, subsequent to or concurrently with ex vivo methods of modifying a subject's hematopoeitic stem cell lineage cells.

Optionally the cell is a somatic cell. Optionally, the cell is a hematopoietic stem cell lineage cell, for example, a T cell. Optionally, the cell is not a hematopoietic stem cell lineage cell, for example, an endothelial cell. Optionally, the cells is any cell that requires secretion of proteins through HLH related pathways For example, a spleen, thymus, lung, placenta, brain, heart, skeletal, muscle or kidney cell. In other examples, in vivo targeting of lymphocytes, cytotoxic T-lymphocytes, and/or natural killer T cells can be performed. Optionally, in vivo targeting of endothelial, liver, intestine, or mesenchymal stem cells and their progency can be performed. In other examples, stem cells derived from in vitro induced pluripotent stem cells (iPSCs) as well as their in vitro and in vivo progeny can be modified. Any of the cell types described herein can be targeted for Munc13-4 or Stxbp2 deficiency. The nucleic acids or vectors described herein can be administered systemically or locally to the subject. Routes of administration include, but are not limited to, parenteral administration, such as subcutaneous, intravenous, or intramuscular injection or infusion.

Optionally, the nucleic acid is delivered as naked DNA or in a plasmid. Optionally, the nucleic acids or vectors described herein are delivered to the subject via nanoparticle delivery. See, for example, U.S. Pat. No. 9,737,604; Zhang et al. “Lipid nanoparticle-mediated efficient delivery of CRISPR/Cas9 for tumor therapy,” NPG Asia Materials Volume 9, page e441 (2017); and Miller “Nanoparticles improve economic mileage for CARs,” Science Translational Medicine 9(387), eaan2784 DOI: 10.1126/scitranslmed.aan2784. In some embodiments, the constructs can be targeted to endogenous immune cell subsets in the circulation via in vivo targeted nanoparticle delivery. See, for example, Schmid et al. “T cell targeting nanoparticles focus delivery of immunotherapy to improve antitumor immunity,” Nature Communications 8, Article Number: 1747 (2017) doi: 10.1038/s41467-017-01830-8.

Optionally, the nucleic acids or vectors are delivered to the subject via direct injection of the subject with an adenoviral, adeno-associated-virus (AAV) vector, retroviral vector or lentiviral vector comprising an expression-optimized nucleic acid sequence encoding a Munc13-4 or STXB2 polypeptide. Optionally, the viral vector is targeted to a cell of interest, for example, by receptor-targeted delivery. See, for example, Buchholz et al. “Surface-Engineered Viral Vectors for Selective and Cell Type-Specific Gene Delivery,” Trends in Biotechnology 33(12): 777-790 (2015)).

In other cases, targeted nuclease in vivo modification of cells in the subject can also be performed. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017).

Optionally, any of the ex vivo or in vivo methods provided herein can be used in combination with one or more therapeutic agents or treatments for HLH. For example, any of the methods described herein can be combined with chemotherapy, immunotherapy (for example, etoposide, cyclosporin A or methotrexate), steroids, antibiotics or antivirals, to name a few.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and in utero subjects, whether male or female, are intended to be covered. Pediatric subjects include subjects ranging in age from birth to eighteen years of age. Thus, pediatric subjects of less than about 10 years of age, five years of age, two years of age, one year of age, six months of age, three months of age, one month of age, one week of age or one day of age are also included as subjects. As used herein, patient or subject may be used interchangeably and can refer to a subject with or at risk of developing a disorder. The term patient or subject includes human and veterinary subjects.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein. In describing and claiming the present disclosure, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article.

As used herein, the term “autologous” is meant to refer to any material derived from the same individual to which it is later to be re-introduced into the individual.

The term “autologous promoters” refers to the same region of DNA that the promoter naturally belongs to, and part of the same gene. “Heterologous promoters” refer to a transfected gene such that the promoter of the transgene is not the autonomous promoter, thus, not naturally associated with the gene.

As used herein, the term “Cas9” refers to an RNA-mediated nuclease (e.g., of bacterial origin, or derived therefrom). Exemplary RNA-mediated nucleases include the foregoing Cas9 proteins and homologs thereof. Other RNA-mediated nucleases include Cpf1 (See, e.g., Zetsche et al. (2015) Cell 163:759-71) and homologs thereof. Similarly, as used herein, the term “Cas9 ribonucleoprotein” complex and the like refers to a complex between the Cas9 protein, and a crRNA (e.g., guide RNA or single guide RNA), the Cas9 protein and a trans-activating crRNA (tracrRNA), the Cas9 protein and a guide RNA, or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA). Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi et al. (2013) RNA Biol. 10(5): 726-37; Hou et al. (2013) Proc. Natl. Acad. Sci. USA 110(39):15644-9; Sampson et al. 2013 Nature 497(7448):254-57; and Jinek et al. (2012) Science 337(6096):816-21. Variants of any of the Cas9 nucleases provided herein can be optimized for efficient activity or enhanced stability in the host cell. Thus, engineered Cas9 nucleases are also contemplated. See, for example, “Slaymaker et al. (2016) “Rationally engineered Cas9 nucleases with improved specificity,” Science 351 (6268): 84-88 (2016)).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s). As used herein “endogenous” refers to any material from or produced inside an organism, cell, tissue or system.

As used herein, the term “exogenous” refers to any material introduced from or produced outside an organism, cell, tissue or system.

As used herein in reference to nucleic acids or amino acids, a percent “identity” refers to alignment and comparison of nucleotide or amino acid residues in one sequence as compared to a reference sequence. One of skill will recognize that these values can be appropriately adjusted to determine corresponding identity of proteins encoded by two nucleotide sequences by taking into account codon degeneracy, amino acid similarity, reading frame positioning and the like. Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff (1989) Proc. Natl. Acad. Sci. USA 89:10915). The BLAST algorithm also performs a statistical analysis of the similarity between two sequences (see, e.g., Karlin & Altschul (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

The term “isolated” refers to separating from the natural environment. With regard to cells, it is intended to include products further expanding the isolated cells. For example, a cell naturally present in a living animal is not “isolated,” but the same cell partially or completely separated from the coexisting materials of its natural state is “isolated.” An isolated nucleic acid or protein can exist in substantially purified form, or can exist in a non-native environment such as, for example, a host cell. In another non-limiting example, a cell removed from a subject is “isolated”.

The term “expand” as used herein refers to increasing in number, as in an increase in the number of cells. In one embodiment, the cells that are expanded ex vivo increase in number relative to the number originally present in the culture. In another embodiment, T cells or hematopoietic cells that are expanded ex vivo increase in number relative to other cell types in the culture.

The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, a hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit+ and lin−. In some cases, human hematopoietic stem cells are identified as CD34+, CD59+, Thy1/CD90+, CD38lo/−, C-kit/CD117+, lin−. In some cases, human hematopoietic stem cells are identified as CD34−, CD59+, Thy1/CD90+, CD38lo/−, C-kit/CD117+, lin−. In some cases, human hematopoietic stem cells are identified as CD133+, CD59+, Thy1/CD90+, CD38lo/−, C-kit/CD117+, lin−. In some cases, mouse hematopoietic stem cells are identified as CD34lo/−, SCA-1+, Thy1+/lo, CD38+, C-kit+, lin−. In some cases, the hematopoietic stem cells are CD150+CD48-CD244-.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein, the phrase “modifying” in the context of modifying a genome of a cell refers to inducing a structural change in the sequence of the genome at a target genomic region. For example, the modifying can take the form of inserting a nucleotide sequence into the genome of the cell. For example, a nucleotide sequence encoding a polypeptide can be inserted into the genomic sequence encoding an endogenous cell surface protein in the T cell. The nucleotide sequence can encode a functional domain or a functional fragment thereof. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region.

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements. Nucleic acids include deoxyribonucleic acids (DNA) or ribonucleic acids (RNA) and polymers thereof.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used throughout, by “subject” is meant an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder.

As used herein, the phrase “T cell” refers to a lymphoid cell that expresses a T cell receptor molecule. T cells include human alpha beta (43) T cells and human gamma delta (γ6) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can also be CD4⁻, CD8⁻, or CD4⁻ and CD8⁻. T cells can be helper cells, for example helper cells of type T_{H}1, T_{H}2, T_{H}3, T_{H}9, T_{H}17, or T_{FH}. T cells can be cytotoxic T cells. Regulatory T cells can be FOXP3⁺ or FOXP3⁻. T cells can be alpha/beta T cells or gamma/delta T cells. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8⁺CD28−, Treg17, and Qa-1 restricted T cells, or a combination or sub-population thereof. In some cases, the T cell is a FOXP3⁺ T cell. In some cases, the T cell is a CD4⁺CD25^{lo} CD127^{hi} effector T cell. In some cases, the T cell is a CD4⁺CD25^{lo} CD127^{hi}CD45RA^{hi}CD45RO⁻ naïve T cell. A T cell can be a recombinant T cell that has been genetically manipulated.

As used herein the terms “treatment,” “treat,” or “treating” refers to a method of reducing one or more of the effects of the disorder or one or more symptoms of the disorder, for example, HLH. Thus in the disclosed method, treatment can refer to a 10%, 20%, 30%, 40%, 50%, 60%, 70%, 80%, 90%, or 100% reduction in the severity of HLH. For example, a method for treating HLH is considered to be a treatment if there is a 10% reduction in one or more symptoms of the HLH in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

The terms “variant” and “mutant” when used in reference to a polypeptide refer to an amino acid sequence that differs by one or more amino acids from another, usually related polypeptide. Variants may be in the form of functioning fragments. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline, and other mammals. Preferably, the subject is human.

As used herein, the term “combination with” when used to describe administration with an additional treatment means that the agent may be administered prior to, together with, or after the additional treatment, or a combination thereof.

“Effective amount” or “therapeutically effective amount” are used interchangeably herein, and refer to an amount of a compound, formulation, material, or composition, as described herein effective to achieve a particular biological result or provides a therapeutic or prophylactic benefit.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

### EXAMPLES

### Materials and Methods

### Cloning

All cloning was done into the LTG 1337 backbone, supplied by Expression Therapeutics (Tucker, Ga.). In humans the UNC13D is comprised of 32 exons. Therefore, in constructing a UNC13D lentiviral vector, the sequence from the Homo sapiens UNCc-13homolog D (UNC13D) mRNA (NCBI reference Sequence: NM_199242.2) was used. When translated, this nucleotide sequence yields isoform 1 of the Munc13-4 protein (protein Sequence NP_954712.1, UniProt identifier Q70J99-1). Codon optimization was performed by GenScript according to the algorithm described in Brown et al. (2018) “Target-Cell-Directed Bioengineering Approaches for Gene Therapy of Hemophilia A,” Mol. Ther. Methods Clin. Dev. 9:57-69. All clones were screened using restriction digests and sequenced by Genewiz (Plainfield, N.J.).

### Lentiviral Vector Packaging and Titering

Third generation, self-inactivating, VSV-G pseudotyped lentiviral vectors were constructed using an HEK 293T transfection protocol according to Johnston et al. (2013) “Generation of an optimized lentiviral vector encoding a high-expression factor VIII transgene for gene therapy of hemophilia A,” Gene Ther. 20(6):607-15. Specifically, HEK 293 T cells were transfected with a four plasmid system consisting of the cloned, transgene-expressing plasmid and Lentigen's plasmids pLTG1292 VSVG, pkREV, and pkGAG-POL in a 3:1:1:1 ratio. The following day the media was replaced with DMEM-F12 supplemented with 10% FBS and 1% penicillin/streptomycin. Supernatant from these transfected cells were collected on days two and three post-transfection. Viral vector was pelleted out of the supernatant by overnight ultracentrifugation at 10,000 g, 4° C. Pelleted viral vector was subsequently filtered through a 0.22 micron filter and re-suspended in StemPro media. All lentiviral vector preparations were titered by applying polybrene (aka “hexadimethrene bromide,” 8 μg/mL) (Sigma, St. Louis. Mo.) and 3, 9, or 27 μl of the new lentiviral vector onto HEK 293T cells, incubating overnight, and then culturing in fresh media for five days. DNA was subsequently isolated from these cells using a Qiagen DNA Micro kit (Qiagen 56304) (Hilden, Germany). Quantitative PCR was performed using Power SYBR Green Master Mix (ThermoFisher 4367659) (Waltham, Mass.) with RRE primers (Forward primer: TGG AGT GGG ACA GAG AAA TTA ACA (SEQ ID NO:9), Reverse primer: GCT GGT TTT GCG ATT CTT CAA (SEQ ID NO:10)) to determine the average number of copies of viral vector DNA that were integrated per cell.

### Transduction

All transductions were done in the presence of polybrene-supplemented media (8 μg/mL, Sigma). Cell lines used in in vitro experiments were transduced once with a single overnight application of lentiviral vector. HSCs that were used for transplant studies were transduced twice, with the first hit of virus being incubated on the cells overnight and the second hit of virus lasting for six hours.

### Transplants

Whole bone marrow was flushed from the tibias and fibias from donor Jinx (“Unc13dJinx,” unc-13 homolog D, Jackson Laboratories (Bar Harbor, Me.)) or CD45.1+ mice. Sca-1⁺ cells were isolated according to manufacturer protocols using Sca-1 antibody (BD Biosciences 557404 (San Jose, Calif.)), biotin-labeled magnetic beads (MACS Miltenyi Biotec 130-090-485 (Bergisch Gladbach, Germany)), and MACS magnetic separation unit (Miltenyi Biotec magnet and stand 130-042-303 and 130-042-109, respectively). These isolated Sca-1 cells were then cultured in stimulation media consisting of StemPro nutrient-supplemented media (Gibco 10640-019), recombinant mouse IL-3 (20 ng/mL; R & D 403-ML), recombinant human IL-11 (100 ng/mL; R & D 218-IL), recombinant human Flt3/Fc (100 ng/mL; R & D 368-ST), mouse mCSF (100 ng/mL; R&D 455 MC), L-glutamine (HyClone SH30034.01 (UT)), and penicillin and strepatividn (Lonza, 09-757F (Basel, Switzerland)). These cells were subsequently transduced with the specified lentiviral gene therapy vector. Prior to transplant, recipient mice were kept on antibiotic water for one week and were lethally irradiated using two 550 rad doses of gamma radiation. For the transplant, recipient mice received one million transduced Sca-1 cells via retro-orbital injection and kept on antibiotic water for two weeks post-transplant.

### Viral Copy Number (VCN) Analysis

The same RRE primers, SYBR Green Power PCR Master Mix, and PCR protocol that were used for the titering of new lentiviral vectors were also used in the qPCR-based copy number analysis of transduced cells.

### Infection

Jinx mice were infected via intraperitoneal injection with 2×10⁵ PFU of LCMV Armstrong.

### CFU Assays

One thousand cells/mL of Methocult Culture media (StemCell Technologies 03434 (Vancouver, Canada)) were plated and incubated for 7-10 days. Colonies were subsequently counted, assessed using a viability stain, and the number of successfully transduced CFUs was determined either by copy number assay or flow cytometry on the pooled cells.

### Western Blot

Western blot analysis was performed by blotting onto a PVDF membrane (BioRad 162-0261 (Hercules, Calif.)) and staining for Munc13-4 (Abcam, Clone ERP4914, Cat #ab109113 (Cambridge, United Kingdom)), and Beta-actin (GenScript, Clone 2D1D10, Cat #A00702 (Piscataway, N.J.)). Secondary antibodies (IRDye 800 CW and IRDye 680, respectively Li-Cor catalog numbers 926-32213 and 026-68072 (Lincoln, Nebr.)) were used to clearly distinguish between the Munc13-4 and beta-actin bands, and the blots were imaged using a Li-Cor Odyssey CLx imaging device using Image Studio Version 4.0 software.

### Degranulation Assay

Homogenized splenic tissue was passed through a 70 μm filter, washed with PBS containing 5% FBS, and plated at 300,000 cells/FACS tube in RPMI containing 10% FBS and 1% Penicillin and Streptavidin and supplemented with mouse IL-2 (10 ng/mL, Biolegend, Cat #575402 (San Diego, Calif.)). Cells were then incubated for 30 minutes in either the presence or absence of 3 μg/mL of CD3 (BioLegend Clone 145-2C11, Cat #100301) and 1 μg/mL of CD28 antibody (Clone 37.51, BioLegend, Cat #102101) to produce “stimulated” and “unstimulated” sample groups respectively. Subsequently, monensin (Biolegend Cat #420701) and CD107a antibody (BV421, BD, Cat #121617) were added to all of the cultures, and the cells were incubated for an additional eight hours. Staining for analysis consisted of a live dead cell stain, CD8 (PE, BioLegend, Cat #108739), CD44 (UV379, BD, Cat #740215 (San Jose, Calif.)), and CD69 (FITC, BD, Cat #557392). The chimeric transplant studies also incorporated the use of CD45.1 and CD45.2 antibodies (PerCP-Cy5.5, BD, Cat #560580 and BUV737, BD, Cat #564880 respectively) to distinguish between cells derived from Jinx and CD45.1 engrafted Sca-1 cells.

### Cytotoxicity Assay

Frozen PBMCs from an FHL3 patient were thawed and stimulated using CD3 and CD28 (7 μg/mL and 0.5 μg/mL, respectively) for 24 hours. These cells were subsequently double transduced with an MOI of 10 for both TPO-CAR and UNC13D lentiviral vectors according to the aforementioned transduction protocol. Five days post-transduction, these cells were co-incubated with M-07e cells. Cell killing was assessed by flow cytometry by looking for cell death markers Annexin V and phosphatidylinositol (PI).

### Cell Lines

The following cell lines were used in these studies: NIH 3T3 cells (ATCC number CRL-1658), Jurkat clone E6-1 cells (ATCC number TIB-152), M-07e cells (ACC 104).

### Cell Culture

The following reagents were used for cell culture: DMEM (Corning, 10-017-CV (Corning, N.Y.)), RPMI (Corning, 10-040-CV), StemPro (Gibco 10640-019), FBS (Atlanta Biologicals, S11150H), penicillin/streptomycin (Lonza, 09-757F).

### Flow Cytometry

The following reagents were used throughout the various flow cytometry based experiments: eFluor 780 viability staining (ThermoFisher 65-0865-14), PE mCD8 (BioLegend 108739), UV379 mCD44 (BD 740215), FITC mCD69 antibody (BD 557392), BV421 mCD107a (BD 121617), PerCP-Cy5.5 CD45.1 (BD 560580), BUV737 CD45.2 (BD 564880), Annexin V and phosphatidylinositol.

### Statistical Analysis

All statistical tests were performed using GraphPad Prism Version 8 (San Diego, Calif.).

### Results

### Transduction of FHL3 Patient T Cells

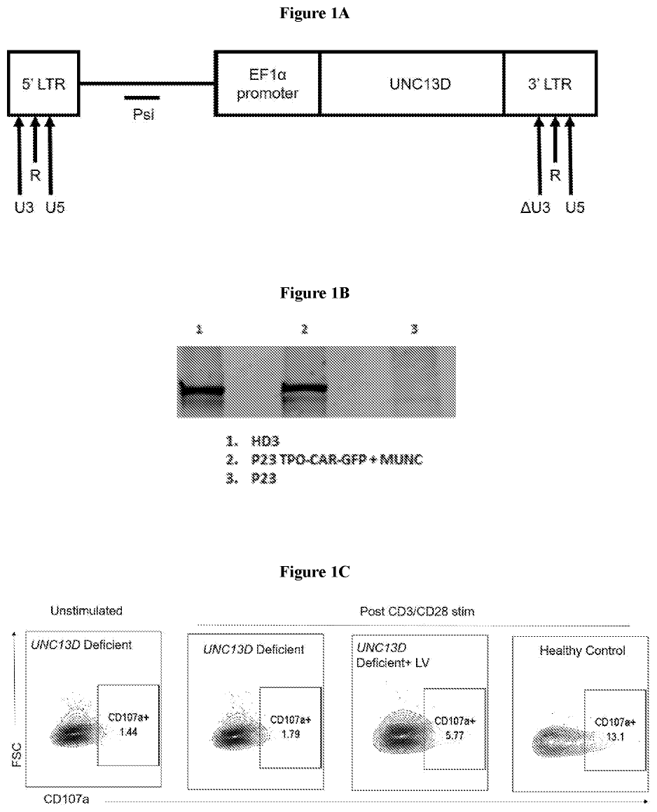

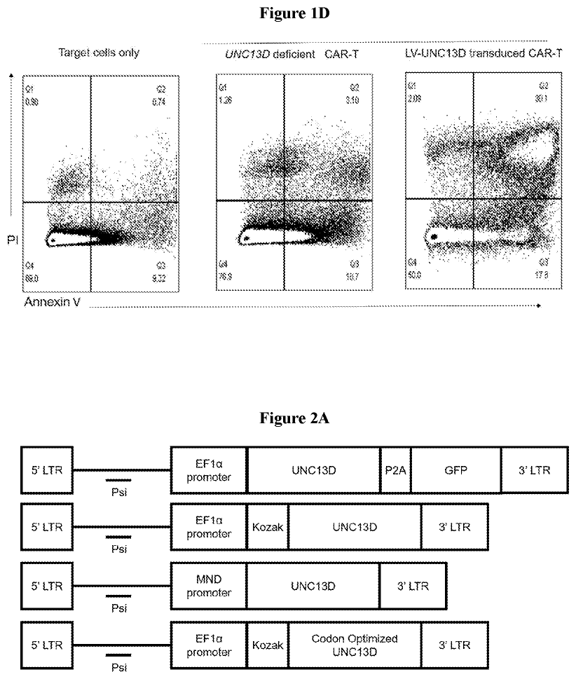

A lentiviral construct that expresses the human UNC13D gene (GenBank Accession Number AJ578444.1 (mRNA)) was designed under the control of the Ef1α promoter (FIG. 1A). Stimulated PBMCs from an FHL3 patient were transduced, and it was observed that these transduced cells expressed the Munc13-4 transgene (FIG. 1B). Stimulation of the transduced cells with CD3 and CD28 induced increased expression of the exocytosis marker CD107a (LAMP-1) at the plasma membrane as compared to non-transduced patient cells, indicating functionality of the transgenic Munc13-4 protein (FIG. 1C). Finally, in order to assess the killing abilities of the transduced cells, FHL3 patient PBMCs were double transduced with both the UNC13D construct as well as a TPO-CAR construct. This respectively rendered the double-transduced cells cytotoxically competent and able to recognize cells expressing the thrombopoietin receptor (MPL). A cytotoxicity assay performed by co-culturing these cells with MPL expressing target cells demonstrated an increase in cytotoxic function in transduced patient cells compared to non-transduced patient cells (FIG. 1D).

### Optimization of the Lentiviral Construct

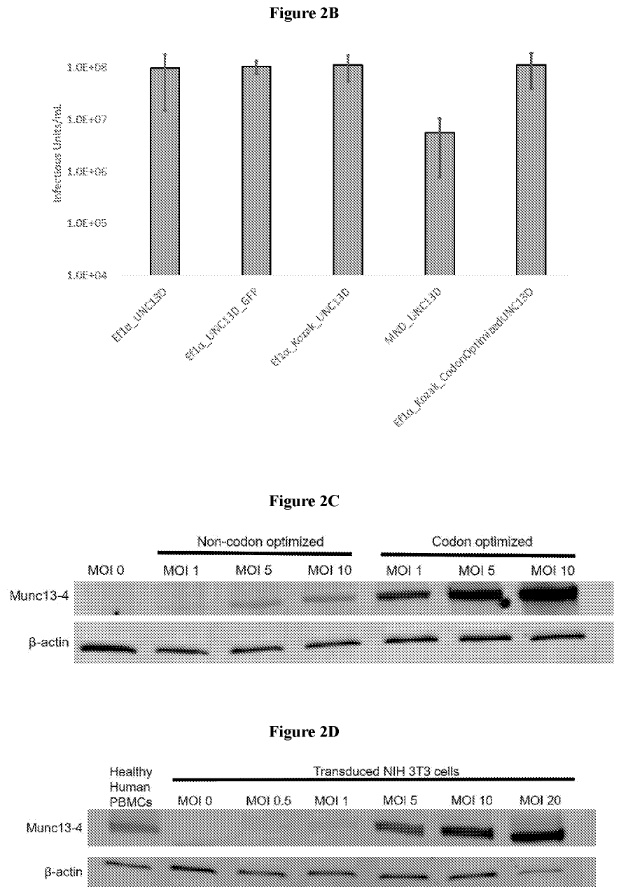

In order to increase the expression of Munc13-4 protein several alternative lentiviral vector-based constructs were designed (FIG. 2A). First, the sequence for green fluorescent protein was added as a marker for transduced cells. Second, the Ef1α promoter was replaced with the synthetic MND promoter, which was adapted from Moloney murine leukemia virus and myeloproliferative sarcoma virus elements. See Li et al. (2010) “Optimal promoter usage for lentiviral vector-mediated transduction of cultured central nervous system cells,” J. Neurosci. Methods 189:56-64. Third, a Kozak sequence was included to enhance translation of transgene mRNA. See Kozak (1986) “Point mutations define a sequence flanking the AUG initiator codon that modulates translation by eukaryotic ribosomes,” Cell 44(2):283-92. Finally, a codon optimized version was created. See Brown (2016) “Bioengineering Viral Transgenes for the Treatment of Hemophilias,” in Molecular and Systems Pharmacology, Emory University. All cloned plasmids were checked for their accuracy using restriction digests and sequencing.

Self-inactivating lentiviral vectors at similar titers (FIG. 2B) were reliably produced using an HEK 293T transfection protocol. See Johnston et al. (2014) “High-throughput screening identifies compounds that enhance lentiviral transduction,” Gene Ther. 21(12):1008-20. These vectors could be used to transduce NIH 3T3 cells (a mouse embryonic fibroblast cell line that does not express Munc13-4 protein) and to robustly express Munc13-4 protein (FIGS. 2C and 2D). The greatest levels of protein expression were produced from cells that were transduced with the Ef1α_Kozak_Codon-Optimized-UNC13D construct, which was used for subsequent transduction experiments.

### Identifying Potentially Therapeutic Munc13-4 Expression Levels

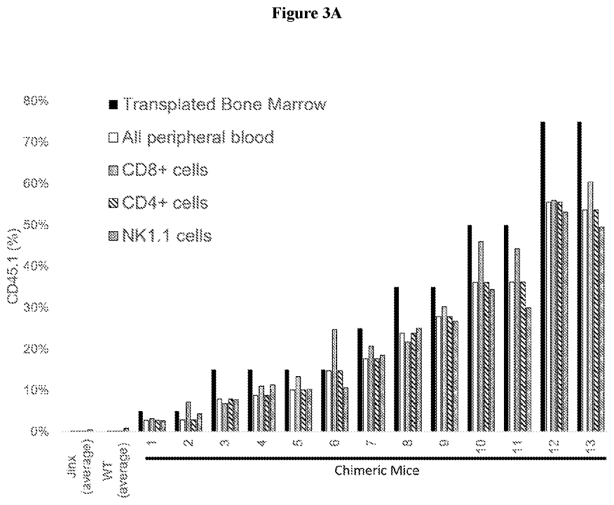

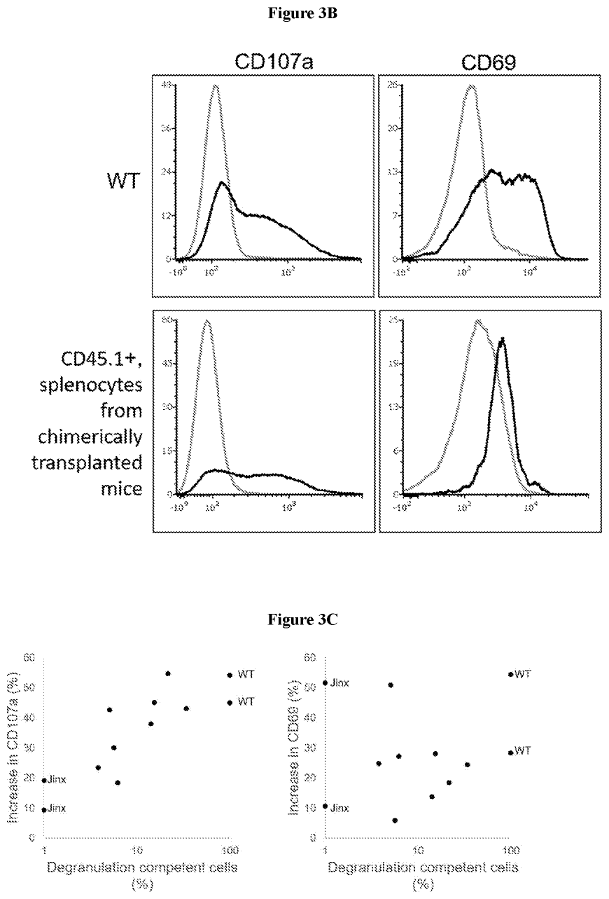

Jinx mice were irradiated twice with 550 rads of gamma radiation and transplanted with different mixtures of Jinx whole bone marrow (CD45.2+) and WT bone marrow (CD45.1+). Flow cytometry analysis showed that the percent composition for each of these bone marrow types were consistent with those of the peripheral blood six weeks after transplant, as well as the specific CD8+, CD4+, and NK1.1+ cell populations (FIG. 3A), indicating Jinx bone marrow engrafted similarly to wildtype bone marrow. Twenty-two weeks post-transplant, the mice were sacrificed and their splenocytes were stimulated with CD3 and CD28 and analyzed for the expression of plasma membrane-bound CD107a (Lamp-1) and CD69. Within the chimeric mice, CD45.1+ bone marrow degranulation remained consistent with that from WT mice, indicating that the engrafted cells were immunocompetent (FIG. 3B). Mice with 15% of CD45.1+ mouse bone marrow showed a degranulation phenotype comparable to that of a WT mouse (FIG. 3C). Together this shows that, similar to the results demonstrated in the FHL2 gene therapy mouse model, a 15% correction should be sufficient to recapitulate the WT phenotype in FHL3 disease model mice. As expected, CD69 expression was similar in all mice, as the activation of these cells is not effected.

Gene Transfer into the FHL3 Disease Mouse Model

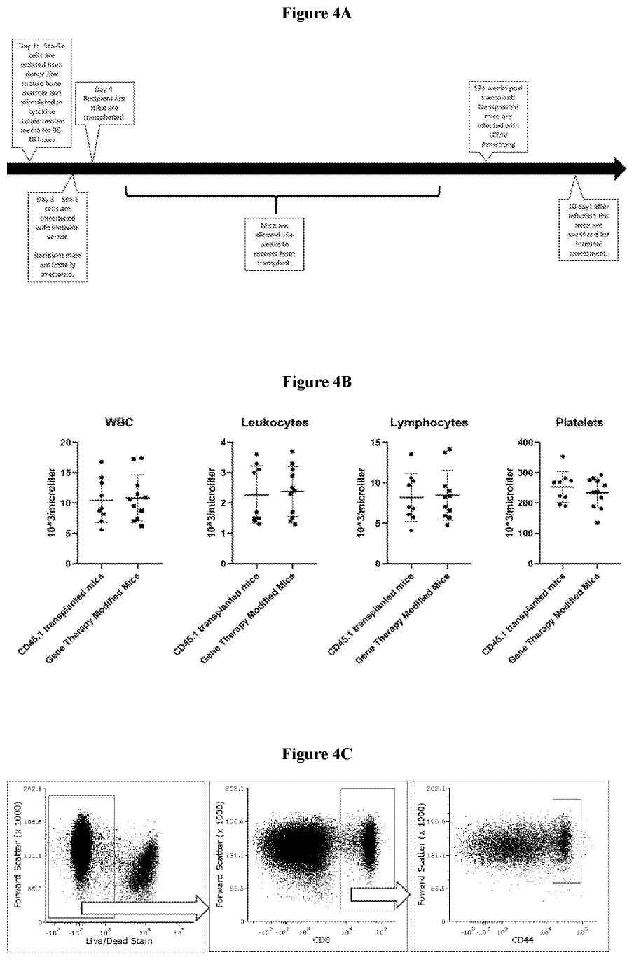

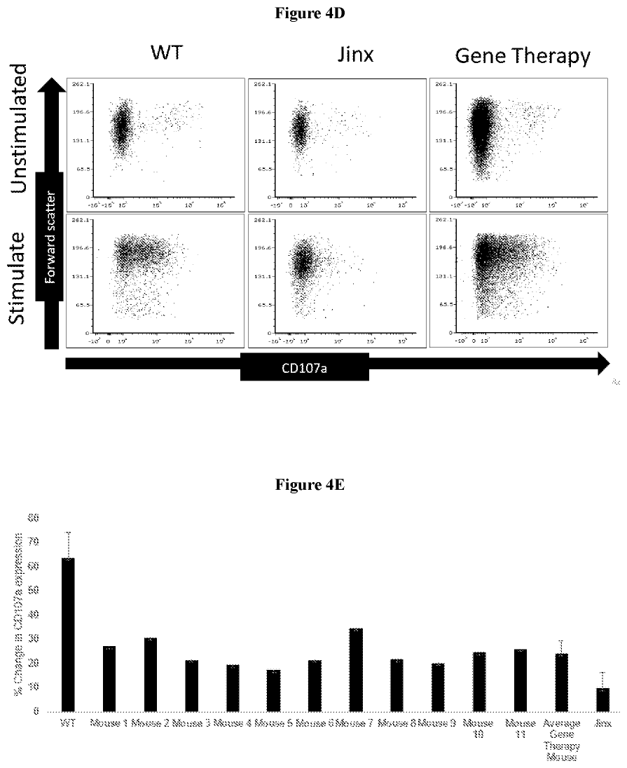

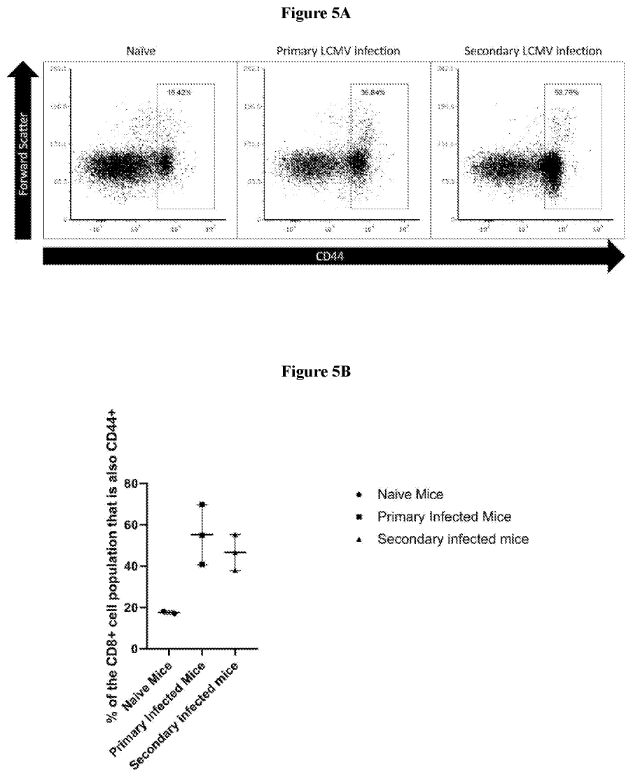

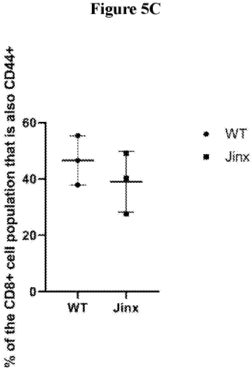

To determine if the codon-optimized construct was effective in vivo, Sca-1 cells were isolated from donor Jinx mice and transduced with our recombinant lentiviral vector, and transplanted into Jinx mice (FIG. 4A). See Tran et al. (2017) “Microfluidic Transduction Harnesses Mass Transport Principles to Enhance Gene Transfer Efficiency,” Mol. Ther. 25(10):2372-82. Within twelve weeks, the transplanted mice showed engraftment and normal complete blood counts (CBCs) (FIG. 4B). Twenty weeks after transplant, the mice were infected with 2×10⁵ PFU LCMV Armstrong in order to induce the FHL3 disease phenotype (FIGS. 5C and 5D). Ten days after infection, the mice were sacrificed, and the degranulation capacity of their CD44+CD8+ T cells was quantified using the previously described degranulation assay (FIG. 5E). It is shown that the mice transplanted with genetically modified bone marrow stem cells had an increase in CD107a expression, indicating enhanced degranulation compared to non-modified mice.

### Benchmarking FHL3 Gene Therapy Goals

Additional experiments were performed to determine what percent correction in a Munc13-4 null mouse model (Jinx mouse model) would be sufficient to correct the Munc13-4 deficient phenotype. To this end, different mixtures of WT (CD45.1) and Jinx (CD45.2) bone marrow were transplanted into lethally irradiated Jinx mice. This produced a gradient of intermediately degranulation-competent mice that maintained their relative proportions of WT vs. Jinx descendent cells across the CD8+, CD4+, and NK1.1+ cell compartments (FIG. 1A). Induction of the FHL3 disease model and subsequent interrogation of splenocytes using a degranulation assay revealed that engrafted CD45.1+ cells could up-regulate CD107a expression to their cell surfaces just as well as splenocytes from non-transplanted WT mice (FIG. 3B), which allowed for the assumption that any increases in CD107a expression could be attributed to an increased proportion of CD45.1 cells. Subjecting bulk splenocytes to the same degranulation assay showed a positive correlation between the proportion of engrafted CD45.1 cells and degranulation capacity (FIG. 3C). No correlation was observed between the proportion of engrafted CD45.1 cells and cell activation (indicated by plasma membrane-bound CD69), which is to be expected given that both CD45.1 and Jinx cells are capable of activation in the presence of CD3 and CD28 antibody (but not degranulation, which is the function of Munc13-4). While the overall trends of this experiment are not surprising, what is important to note is the point at which the degranulation of the mixed chimera mice begins to rival that of WT mice. This happens when about 15% of the peripheral blood consists of WT cells. This result is consistent with the findings of FHL2 gene therapy research as well as clinical findings. Extrapolating this finding to FHL3 gene therapy research, it is therefore feasible that genetically modifying as little as 15% of the total number of circulating cells could correct the FHL3 phenotype. This assumes that each transduced cell will be able to function as well as a WT cell with regards to serial cell killing.

Gene Transfer into the FHL3 Disease Mouse Model

Having established an attainable benchmark goal for the percent of gene corrected HSCs needed to engraft in a Jinx mouse, the lentiviral vector was used to supplement Jinx HSCs with a working copy of the UNC13D gene. Lethally irradiated mice received one million Sca-1 cells transduced with the UNC13D lentiviral vector. By twelve weeks post-transplant these mice had normal complete blood counts compared to control Jinx mice that had received one million CD45.1 cells (FIG. 4B). Of particular importance, gene therapy modified mice exhibited similar platelet levels compared to CD45.1 transplanted mice, indicating no increased risk of thrombocytopenias as a result of transplant. This is particularly important because Jinx mice are more likely to develop thrombocytopenia and thrombocytopenia has been identified as a key prognostic factor in multiple studies of HLH patient outcomes.

After twelve weeks post-transplant, the mice were injected with LCMV Armstrong in order to induce the well-characterized FHL3 disease phenotype. Ten days after infection these mice had increased numbers of CD44+ cells, indicating the expansion of memory T cells within the infected mice (FIG. 5A-C). For degranulation assays, changes in CD107a expression within these cells were observed because these changes represent the populations that recognized and responded to the LCMV challenge (FIG. 4C though 4E). Upon stimulation, these cells showed significantly greater expression of CD107a compared to Jinx mice that had not been gene therapy modified. CD107a expression on cytotoxic cells is both an important diagnostic marker for FHL3 patients and an indicator of cytolytic ability using the perforin/granzyme pathway. Therefore the restoration of CD107a expression in degranulation assays indicated successful gene therapy modification and potential for FHL3 disease correction.

LCMV Armstrong Infection in Mice Induces a Robust Immunological Response Consistent with Previous Descriptions of the FHL3 Mouse Model

C57BL/6 mice were infected with 2×10⁵ PFU of LCMV Armstrong via intraperitoneal injection. Seven days after injection, the numbers of CD44+CD8+ cells had increased compared to LCMV-naïve mice as analyzed by flow cytometry (FIG. 5A, left and center panels). A subsequent second injection of 2×10⁵ PFU of LCMV Armstrong increased this immune response even more, though not enough to warrant administering secondary injections throughout the course of this study (FIG. 5A, right panel and FIG. 5B). Administering a single dose of LCMV Armstrong to Jinx mice induced a similar immune response (FIG. 5C). Together, these data validate the viral stocks induced the FHL3 disease model.

### CONCLUSION

Codon optimization results in an increase in transgene expression. The differences in protein expression were not due to differences in transgene copy number. Optimized UNC13D lentiviral vector can be used to effectively modify FHL3 patient T cells as well as hematopoietic stem cells from the FHL3 (Jinx) mouse model. Furthermore, these data indicate that, if as few as 15% of the circulating CD8+ T cells can be modified, the FHL3 disease phenotype can be resolved.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

Expression-Optimized UNC13D nucleic acid sequence
SEQ ID NO: 1
ATGGCAACACTCCTCAGTCATCCACAGCAGAGACCTCCTTTCCTCAGACAGGCAA
TCAAAATCAGACGGCGGCGGGTGCGGGACCTCCAGGATCCCCCACCCCAGATGG
CCCCAGAAATCCAGCCACCCAGCCACCACTTCTCCCCCGAACAGAGGGCTCTGCT
GTACGAGGACGCCCTGTACACCGTGCTGCACAGACTGGGCCACCCAGAGCCCAA
CCACGTGACCGAAGCCAGCGAGCTGCTGAGGTACCTGCAGGAAGCTTTCCACGT
GGAGCCAGAGGAACACCAGCAGACCCTG
CAGAGAGTGAGAGAACTGGAGAAGCCCATCTTCTGCCTGAAGGCTACCGTGAAG
CAGGCCAAGGGCATCCTGGGCAAGGACGTGAGCGGCTTCTCCGATCCCTACTGC
CTGCTGGGCATCGAGCAGGGGGTGGGGGTGCCAGGCGGCAGCCCCGGCTCCAGG
CACAGACAGAAGGCTGTCGTGAGGCACACCATCCCAGAAGAAGAAACACACAG
AACCCAGGTGATCACCCAGACCCTGAACCCCGTGTGGGACGAAACCTTCATCCT
GGAATTCGAGGACATCACCAATGCCAGCTTCCACCTGGACATGTGGGACCTGGA
TACCGTGGAATCCGTGAGGCAGAAGCTGGGCGAGCTGACCGATCTGCACGGCCT
GAGGAGAATCTTCAAGGAAGCTAGGAAGGACAAGGGCCAGGACGATTTCCTGGG
CAACGTGGTGCTGAGACTGCAGGACCTGAGGTGCAGAGAGGATCAGTGGTACCC
ACTGGAACCCAGGACCGAAACCTACCCAGATAGAGGCCAGTGCCACCTGCAGTT
CCAGCTGATCCACAAGAGGAGAGCTACCAGCGCCTCCAGAAGCCAGCCCAGCTA
CACCGTGCACCTGCACCTGCTGCAGCAGCTGGTGTCCCACGAAGTGACCCAGCA
CGAGGCTGGATCCACCAGCTGGGATGGCTCCCTGAGCCCACAGGCTGCTACCGT
GCTGTTCCTGCACGCTACCCAGAAGGACCTGAGCGATTTCCACCAGTCCATGGCC
CAGTGGCTGGCTTACAGCAGGCTGTACCAGTCCCTGGAGTTCCCAAGCTCCTGCC
TGCTGCACCCCATCACCAGCATCGAATACCAGTGGATCCAGGGCAGACTGAAGG
CCGAGCAGCAGGAAGAGCTGGCCGCTTCCTTCAGCTCCCTGCTGACCTACGGCCT
GAGCCTGATCAGGAGATTCAGGTCCGTGTTCCCACTGTCCGTGAGCGATTCCCCC
GCTAGGCTGCAGAGCCTGCTGAGAGTGCTGGTGCAGATGTGCAAGATGAAGGCT
TTCGGCGAACTGTGCCCAAATACCGCCCCACTGCCACAGCTGGTGACCGAAGCTC
TGCAGACCGGCACCACCGAGTGGTTCCACCTGAAGCAGCAGCACCACCAGCCAA
TGGTGCAGGGCATCCCAGAGGCTGGGAAGGCTCTGCTGGGCCTGGTGCAGGACG
TGATCGGCGATCTGCACCAGTGCCAGAGGACCTGGGACAAGATCTTCCACAATA
CCCTGAAGATCCACCTGTTCAGCATGGCCTTCAGGGAGCTGCAGTGGCTGGTGGC
TAAGAGAGTGCAGGACCACACCACCGTGGTGGGCGATGTGGTGAGCCCAGAAAT
GGGCGAGTCCCTGTTCCAGCTGTACATCAGCCTGAAGGAACTGTGCCAGCTGAG
GATGAGCAGCTCCGAGAGAGATGGGGTGCTGGCTCTGGATAACTTCCACAGGTG
GTTCCAGCCAGCTATCCCCAGCTGGCTGCAGAAGACCTACAATGAGGCTCTGGCC
AGGGTGCAGAGAGCCGTGCAGATGGATGAACTGGTGCCCCTGGGCGAGCTGACC
AAGCACAGCACCTCCGCTGTGGACCTGAGCACCTGCTTCGCTCAGATCTCCCACA
CCGCTAGGCAGCTGGACTGGCCAGATCCAGAAGAGGCCTTCATGATCACCGTGA
AGTTCGTGGAAGACACCTGCAGACTGGCTCTGGTGTACTGCAGCCTGATCAAGGC
TAGGGCCAGAGAGCTGTCCTCCGGACAGAAGGATCAGGGACAGGCTGCTAACAT
GCTGTGCGTGGTGGTGAATGACATGGAGCAGCTGAGGCTGGTGATCGGAAAGCT
GCCAGCTCAGCTGGCTTGGGAAGCTCTGGAGCAGAGGGTGGGGGCTGTGCTGGA
ACAGGGACAGCTGCAGAACACCCTGCACGCTCAGCTGCAGAGCGCTCTGGCTGG
CCTGGGACACGAGATCAGGACCGGCGTGAGAACCCTGGCTGAACAGCTGGAAGT
GGGCATCGCCAAGCACATCCAGAAGCTGGTGGGCGTGAGGGAAAGCGTGCTGCC
AGAGGATGCCATCCTGCCCCTGATGAAGTTCCTGGAAGTGGAGCTGTGCTACATG
AACACCAATCTGGTGCAGGAAAATTTCTCCAGCCTGCTGACCCTGCTGTGGACCC
ACACCCTGACCGTGCTGGTGGAGGCTGCTGCTAGCCAGAGGTCCAGCTCCCTGGC
TTCCAACAGACTGAAGATCGCCCTGCAGAATCTGGAAATCTGCTTCCACGCTGAG
GGATGCGGCCTGCCACCCAAGGCCCTGCACACCGCTACCTTCCAGGCCCTGCAG
AGGGACCTGGAACTGCAGGCTGCCAGCTCCAGGGAGCTGATCAGAAAGTACTTC
TGCAGCAGAATCCAGCAGCAGGCCGAAACCACCTCCGAAGAGCTGGGCGCTGTG
ACCGTGAAGGCTAGCTACAGGGCCTCCGAACAGAAGCTGAGAGTGGAGCTGCTG
AGCGCTAGCTCCCTGCTGCCACTGGACTCCAACGGCAGCTCCGATCCCTTCGTGC
AGCTGACCCTGGAACCAAGGCACGAATTCCCCGAGCTGGCTGCCAGAGAAACCC
AGAAGCACAAGAAGGACCTGCACCCCCTGTTCGATGAAACCTTCGAGTTCCTGGT
GCCCGCAGAACCCTGCAGAAAGGCTGGGGCTTGCCTGCTGCTGACCGTGCTGGA
CTACGATACCCTGGGCGCTGACGATCTGGAAGGCGAGGCCTTCCTGCCCCTGAG
AGAAGTGCCAGGCCTGAGCGGATCCGAAGAACCCGGGGAGGTGCCACAGACCA
GACTGCCACTGACCTACCCAGCCCCCAATGGCGATCCCATCCTGCAGCTGCTGGA
AGGCAGGAAGGGCGACAGAGAGGCTCAGGTGTTCGTCAGACTCAGGAGACATA
GAGCAAAACAGGCATCACAGCACGCCCTCAGACCAGCCCCCTAG
Expression-Optimized STXBP2 nucleic acid sequence
SEQ ID NO: 2
ATGGCACCAAGCGGGCTCAAGGCAGTCGTGGGAGAAAAAATCCTCAGCGGAGTC
ATCAGAAGTGTGAAGAAAGACGGGGAGTGGAAAGTGCTGATCATGGACCACCCC
AGCATGAGAATCCTGAGCTCCTGCTGCAAGATGTCCGACATCCTGGCTGAAGGC
ATCACCATCGTGGAGGACATCAATAAGAGGAGAGAACCCATCCCAAGCCTGGAG
GCCATCTACCTGCTGTCCCCAACCGAGAAGGCTCAGGCTCAGAGAGTGATCCAC
CTGCCACAGAGCGTGCAGGCTCTGATCAAGGATTTCCAGGGCACCCCCACCTTCA
CCTACAAGGCCGCTCACATCTTCTTCACCGACACCTGCCCCGAACCACTGTTCAG
CGAGCTGGGCAGGTCCAGACTGGCCAAGGTGGTGAAGACCCTGAAGGAAATCCA
CCTGGCTTTCCTGCCATACGAGGCCCAGGTGTTCAGCCTGGATGCTCCCCACTCC
ACCTACAATCTGTACTGCCCATTCAGGGCCGAGGAAAGGACCAGACAGCTGGAA
GTGCTGGCCCAGCAGATCGCTACCCTGTGCGCCACCCTGCAGGAATACCCCGCCA
TCAGGTACAGAAAGGGCCCAGAGGATACCGCTCAGCTGGCTCACGCTGTGCTGG
CTAAGCTGAACGCCTTCAAGGCTGACACCCCAAGCCTGGGGGAGGGACCAGAAA
AGACCAGGTCCCAGCTGCTGATCATGGATAGGGCTGCTGACCCCGTGAGCCCAC
TGCTGCACGAGCTGACCTTCCAGGCTATGGCCTACGATCTGCTGGACATCGAACA
GGACACCTACAGATACGAAACCACCGGCCTGTCCGAGGCTAGAGAAAAGGCCGT
GCTGCTGGATGAAGACGATGACCTGTGGGTGGAGCTGAGGCACATGCACATCGC
TGACGTGAGCAAGAAGGTGACCGAACTGCTGAGGACCTTCTGCGAGTCCAAGAG
ACTGACCACCGATAAGGCCAATATCAAGGACCTGAGCCAGATCCTGAAGAAGAT
GCCCCAGTACCAGAAGGAGCTGAACAAGTACTCCACCCACCTGCACCTGGCTGA
TGACTGCATGAAGCACTTCAAGGGCAGCGTGGAAAAGCTGTGCTCCGTGGAGCA
GGATCTGGCTATGGGCAGCGACGCCGAGGGCGAAAAGATCAAGGATTCCATGAA
GCTGATCGTGCCAGTGCTGCTGGATGCTGCTGTGCCAGCTTACGACAAGATCAGG
GTGCTGCTGCTGTACATCCTGCTGAGAAACGGCGTGAGCGAGGAAAATCTGGCC
AAGCTGATCCAGCACGCTAACGTGCAGGCCCACAGCTCCCTGATCAGGAATCTG
GAGCAGCTGGGCGGCACCGTGACCAATCCAGGCGGCAGCGGAACCAGCTCCAGG
CTGGAGCCCAGGGAAAGAATGGAGCCAACCTACCAGCTGTCCAGATGGACCCCC
GTGATCAAGGATGTGATGGAAGACGCCGTGGAGGATAGGCTGGACAGAAATCTG
TGGCCCTTCGTGAGCGACCCCGCTCCAACCGCTAGCTCCCAGGCTGCTGTGTCCG
CTAGATTCGGCCACTGGCACAAGAACAAGGCTGGAATCGAGGCTAGGGCTGGAC
CAAGACTGATCGTGTACGTGATGGGCGGCGTGGCTATGAGCGAAATGAGGGCCG
CTTACGAGGTGACCAGAGCCACCGAGGGCAAGTGGGAGGTGCTGATCGGCAGCT
CCCACATCCTGACCCCAACCAGATTTCTGGACGACCTCAAAGCCCTGGACAAAA
AACTGGAAGACATCGCTCTGCCCTAA
Expression-Optimized Munc13-4 protein sequence
SEQ ID NO: 3
MATLLSHPQQRPPFLRQAIKIRRRRVRDLQDPPPQMAPEIQPPSHHFSPEQRALLYED
ALYTVLHRLGHPEPNHVTEASELLRYLQEAFHVEPEEHQQTLQRVRELEKPIFCLKAT
VKQAKGILGKDVSGFSDPYCLLGIEQGVGVPGGSPGSRHRQKAVVRHTIPEEETHRT
QVITQTLNPVWDETFILEFEDITNASFHLDMWDLDTVESVRQKLGELTDLHGLRRIFK
EARKDKGQDDFLGNVVLRLQDLRCREDQWYPLEPRTETYPDRGQCHLQFQLIHKRR
ATSASRSQPSYTVHLHLLQQLVSHEVTQHEAGSTSWDGSLSPQAATVLFLHATQKDL
SDFHQSMAQWLAYSRLYQSLEFPSSCLLHPITSIEYQWIQGRLKAEQQEELAASFSSL
LTYGLSLIRRFRSVFPLSVSDSPARLQSLLRVLVQMCKMKAFGELCPNTAPLPQLVTE
ALQTGTTEWFHLKQQHHQPMVQGIPEAGKALLGLVQDVIGDLHQCQRTWDKIFHN
TLKIHLFSMAFRELQWLVAKRVQDHTTVVGDVVSPEMGESLFQLYISLKELCQLRMS
SSERDGVLALDNFHRWFQPAIPSWLQKTYNEALARVQRAVQMDELVPLGELTKHST
SAVDLSTCFAQISHTARQLDWPDPEEAFMITVKFVEDTCRLALVYCSLIKARARELSS
GQKDQGQAANMLCVVVNDMEQLRLVIGKLPAQLAWEALEQRVGAVLEQGQLQNT
LHAQLQSALAGLGHEIRTGVRTLAEQLEVGIAKHIQKLVGVRESVLPEDAILPLMKFL
EVELCYMNTNLVQENFSSLLTLLWTHTLTVLVEAAASQRSSSLASNRLKIALQNLEIC
FHAEGCGLPPKALHTATFQALQRDLELQAASSRELIRKYFCSRIQQQAETTSEELGAV
TVKASYRASEQKLRVELLSASSLLPLDSNGSSDPFVQLTLEPRHEFPELAARETQKHK
KDLHPLFDETFEFLVPAEPCRKAGACLLLTVLDYDTLGADDLEGEAFLPLREVPGLS
GSEEPGEVPQTRLPLTYPAPNGDPILQLLEGRKGDREAQVFVRLRRHRAKQASQHAL
RPAP
Expression-Optimized STXBP2 protein sequence
SEQ ID NO: 4
MAPSGLKAVVGEKILSGVIRSVKKDGEWKVLIMDHPSMRILSSCCKMSDILAEGITIV
EDINKRREPIPSLEAIYLLSPTEKAQAQRVIHLPQSVQALIKDFQGTPTFTYKAAHIFFT
DTCPEPLFSELGRSRLAKVVKTLKEIHLAFLPYEAQVFSLDAPHSTYNLYCPFRAEER
TRQLEVLAQQIATLCATLQEYPAIRYRKGPEDTAQLAHAVLAKLNAFKADTPSLGEG
PEKTRSQLLIMDRAADPVSPLLHELTFQAMAYDLLDIEQDTYRYETTGLSEAREKAV
LLDEDDDLWVELRHMHIADVSKKVTELLRTFCESKRLTTDKANIKDLSQILKKMPQY
QKELNKYSTHLHLADDCMKHFKGSVEKLCSVEQDLAMGSDAEGEKIKDSMKLIVPV
LLDAAVPAYDKIRVLLLYILLRNGVSEENLAKLIQHANVQAHSSLIRNLEQLGGTVTN
PGGSGTSSRLEPRERMEPTYQLSRWTPVIKDVMEDAVEDRLDRNLWPFVSDPAPTAS
SQAAVSARFGHWHKNKAGIEARAGPRLIVYVMGGVAMSEMRAAYEVTRATEGKW
EVLIGSSHILTPTRFLDDLKALDKKLEDIALP
Wildtype UNC13 nucleic acid sequence
SEQ ID NO: 5
ATGGCGACACTCCTCTCCCATCCGCAGCAGCGCCCTCCCTTCTTGCGCCAGGCCA
TCAAGATAAGGCGCCGCAGAGTCAGAGATCTACAGGATCCCCCGCCCCAAATGG
CCCCGGAGATCCAGCCTCCATCCCACCACTTCTCCCCCGAGCAGCGGGCCCTGCT
CTACGAGGACGCACTCTACACTGTCTTGCACCGCCTGGGTCATCCTGAGCCCAAC
CATGTGACGGAGGCCTCTGAGCTGCTGCGATACCTGCAGGAGGCCTTCCACGTGG
AGCCCGAGGAGCACCAGCAGACACTGCAGCGGGTCAGGGAGCTTGAGAAGCCA
ATATTTTGTCTGAAGGCAACAGTGAAACAGGCCAAGGGCATTCTGGGCAAAGAT
GTCAGTGGGTTCAGCGACCCCTACTGCCTGCTGGGCATTGAGCAGGGGGTAGGT
GTGCCAGGGGGCAGCCCCGGGTCCCGGCATCGGCAGAAGGCTGTGGTGAGGCAC
ACCATCCCCGAGGAGGAGACCCACCGCACGCAGGTCATCACCCAGACACTCAAC
CCCGTCTGGGACGAGACCTTCATCCTGGAGTTTGAGGACATCACCAATGCGAGCT
TT
CATCTGGACATGTGGGACCTGGACACTGTGGAGTCTGTCCGACAGAAGCTTGGG
GAGCTCACGGATCTGCATGGGCTTCGCAGGATCTTTAAAGAGGCCCGGAAGGAC
AAAGGCCAGGACGACTTTCTGGGGAACGTGGTTCTGAGGCTGCAGGACCTGCGC
TGCCGAGAGGACCAGTGGTACCCCCTGGAACCCCGCACTGAGACCTACCCAGAC
CGAGGCCAGTGCCACCTCCAGTTCCAACTCATCCATAAGCGGAGAGCCACTTCG
GCCAGCCGCTCGCAGCCGAGCTACACCGTGCACCTCCACCTCCTGCAGCAGCTTG
TGTCCCACGAGGTCACCCAGCACGAGGCGGGAAGCACCTCCTGGGACGGGTCGC
TGAGTCCCCAGGCTGCCACCGTCCTCTTTCTGCACGCCACACAGAAGGACCTATC
CGACTTCCACCAGTCCATGGCGCAGTGGCTGGCCTACAGCCGCCTCTACCAGAGC
CTGGAGTTCCCCAGCAGCTGCCTCCTGCACCCCATCACCAGCATCGAGTACCAGT
GGATCCAGGGTCGGCTCAAGGCAGAACAGCAGGAGGAGCTGGCCGCCTCATTCA
GCTCCCTGCTGACCTACGGCCTCTCCCTCATCCGGAGGTTCCGCTCTGTCTTCCCC
CTCTCTGTCTCGGACTCCCCAGCCCGGCTGCAGTCTCTTCTCAGGGTCCTGGTACA
GATGTGCAAGATGAAGGCCTTTGGAGAACTGTGCCCCAACACCGCCCCATTGCC
CCAGCTGGTGACTGAGGCCCTGCAGACTGGCACCACTGAATGGTTCCACCTGAA
GCAGCAGCACCATCAACCCATGGTGCAGGGCATCCCGGAGGCAGGCAAGGCCTT
GCTGGGCCTGGTACAGGATGTCATTGGCGACCTGCACCAGTGCCAGCGCACATG
GGACAAGATCTTCCACAATACCCTCAAGATCCACCTCTTCTCCATGGCTTTCCGG
GAGCTGCAGTGGCTGGTGGCCAAGCGGGTGCAGGACCACACGACGGTTGTGGGT
GATGTAGTGTCCCCAGAGATGGGCGAGAGTCTGTTCCAGCTCTACATCAGCCTCA
AGGAGCTCTGCCAGCTGCGCATGAGCTCCTCAGAGAGGGATGGAGTCCTGGCCC
TGGATAATTTCCACCGCTGGTTCCAGCCGGCCATCCCCTCCTGGCTGCAGAAGAC
GTACAACGAGGCCCTGGCGCGGGTGCAGCGCGCTGTGCAGATGGATGAGCTGGT
GCCCCTGGGTGAACTGACCAAGCACAGCACATCAGCGGTGGATCTATCCACCTG
CTTTGCCCAGATCAGCCACACTGCCCGGCAGCTGGACTGGCCAGACCCAGAGGA
GGCCTTCATGATTACCGTCAAGTTTGTGGAGGACACCTGTCGCCTGGCCCTGGTG
TACTGCAGCCTTATAAAGGCCCGGGCCCGCGAGCTCTCTTCAGGCCAGAAGGAC
CAAGGCCAGGCAGCCAACATGCTGTGTGTGGTGGTGAATGACATGGAGCAGCTG
CGGCTGGTGATCGGCAAGTTGCCCGCCCAGCTGGCATGGGAGGCCCTGGAGCAG
CGGGTAGGGGCCGTGCTGGAGCAGGGGCAGCTGCAGAACACGCTGCATGCCCAG
CTGCAGAGCGCGCTGGCCGGGCTGGGCCATGAGATCCGCACTGGCGTCCGCACC
CTGGCCGAGCAGTTGGAGGTGGGCATCGCCAAGCACATCCAGAAACTGGTGGGC
GTCAGGGAGTCTGTCCTGCCTGAGGATGCCATTCTGCCCCTGATGAAGTTCCTGG
AGGTG
GAGCTTTGCTACATGAACACCAACTTGGTGCAGGAGAACTTCAGCAGCCTCCTGA
CCCTGCTCTGGACCCACACACTCACAGTGCTGGTGGAGGCGGCCGCCTCCCAGCG
CAGCTCATCCCTGGCTTCCAACAGGCTGAAGATTGCCCTGCAGAACCTGGAGATC
TGCTTCCACGCTGAGGGCTGTGGCCTGCCACCCAAGGCCCTGCACACTGCCACCT
TCCAGGCTCTGCAGAGGGACCTGGAGCTGCAGGCGGCCTCCAGCCGGGAACTCA
TCCGGAAGTACTTCTGCAGCCGAATCCAGCAGCAGGCAGAAACCACCTCTGAGG
AGCTGGGGGCTGTGACAGTCAAGGCCTCCTACCGCGCCTCTGAGCAGAAGCTGC
GTGTGGAGCTGCTCAGCGCCTCCAGCCTGCTGCCCCTGGACTCCAATGGCTCCAG
CGACCCCTTTGTCCAGCTGACCTTGGAGCCCAGGCATGAGTTCCCTGAGCTGGCC
GCCCGGGAGACCCAGAAGCACAAGAAGGACCTTCACCCATTGTTTGATGAGACC
TTTGAATTCCTGGTGCCTGCTGAGCCGTGCCGCAAGGCTGGGGCATGCCTCCTGC
TCACCGTGCTGGACTACGACACGCTGGGGGCCGACGACCTGGAAGGCGAGGCCT
TCCTGCCGCTGCGTGAGGTGCCCGGGCTGAGTGGCTCTGAGGAGCCTGGTGAGGT
GCCTCAGACCCGCCTGCCCCTCACGTACCCCGCACCCAACGGGGACCCAATCCTG
CAGCTGCTGGAGGGCCGGAAGGGTGACCGAGAAGCCCAGGTCTTTGTGAGGCTG
CGGCGGCACCGGGCCAAGCAGGCCTCCCAGCATGCCTTGCGGCCGGCACCGTAG
Wildtype Munc13-4 amino acid sequence
SEQ ID NO: 6
MATLLSHPQQRPPFLRQAIKIRRRRVRDLQDPPPQMAPEIQPPSHHFSPEQRALLYED
ALYTVLHRLGHPEPNHVTEASELLRYLQEAFHVEPEEHQQTLQRVRELEKPIFCLKAT
VKQAKGILGKDVSGFSDPYCLLGIEQGVGVPGGSPGSRHRQKAVVRHTIPEEETHRT
QVITQTLNPVWDETFILEFEDITNASFHLDMWDLDTVESVRQKLGELTDLHGLRRIFK
EARKDKGQDDFLGNVVLRLQDLRCREDQWYPLEPRTETYPDRGQCHLQFQLIHKRR
ATSASRSQPSYTVHLHLLQQLVSHEVTQHEAGSTSWDGSLSPQAATVLFLHATQKDL
SDFHQSMAQWLAYSRLYQSLEFPSSCLLHPITSIEYQWIQGRLKAEQQEELAASFSSL
LTYGLSLIRRFRSVFPLSVSDSPARLQSLLRVLVQMCKMKAFGELCPNTAPLPQLVTE
ALQTGTTEWFHLKQQHHQPMVQGIPEAGKALLGLVQDVIGDLHQCQRTWDKIFHN
TLKIHLFSMAFRELQWLVAKRVQDHTTVVGDVVSPEMGESLFQLYISLKELCQLRMS
SSERDGVLALDNFHRWFQPAIPSWLQKTYNEALARVQRAVQMDELVPLGELTKHST
SAVDLSTCFAQISHTARQLDWPDPEEAFMITVKFVEDTCRLALVYCSLIKARARELSS
GQKDQGQAANMLCVVVNDMEQLRLVIGKLPAQLAWEALEQRVGAVLEQGQLQNT
LHAQLQSALAGLGHEIRTGVRTLAEQLEVGIAKHIQKLVGVRESVLPEDAILPLMKFL
EVELCYMNTNLVQENFSSLLTLLWTHTLTVLVEAAASQRSSSLASNRLKIALQNLEIC
FHAEGCGLPPKALHTATFQALQRDLELQAASSRELIRKYFCSRIQQQAETTSEELGAV
TVKASYRASEQKLRVELLSASSLLPLDSNGSSDPFVQLTLEPRHEFPELAARETQKHK
KDLHPLFDETFEFLVPAEPCRKAGACLLLTVLDYDTLGADDLEGEAFLPLREVPGLS
GSEEPGEVPQTRLPLTYPAPNGDPILQLLEGRKGDREAQVFVRLRRHRAKQASQHAL
RPAP
Wildtype STXBP2 nucleic acid sequence
SEQ ID NO: 7
ATGGCGCCCTCGGGGCTGAAGGCGGTGGTGGGGGAAAAAATTCTGAGCGGAGTT
ATTCGGAGTGTCAAGAAGGATGGGGAGTGGAAGGTGCTTATCATGGATCACCCA
AGCATGCGCATCTTGTCTTCCTGCTGCAAAATGTCAGATATCCTGGCTGAGGGCA
TCACCATTGTTGAAGACATCAACAAACGGCGGGAACCCATTCCCAGTCTGGAGG
CCATTTATTTGCTGAGCCCCACGGAGAAGGCTCAGGCCCAGAGAGTGATCCACCT
TCCCCAGTCGGTTCAGGCCCTGATCAAAGACTTCCAGGGGACCCCGACTTTCACC
TACAAAGCGGCCCATATCTTCTTCACCGACACCTGCCCCGAGCCCCTGTTCAGTG
AGCTAGGCCGCTCTCGTCTGGCAAAGGTGGTGAAGACGTTGAAGGAGATTCACC
TTGCCTTCCTCCCCTACGAGGCCCAGGTGTTCTCCCTCGATGCTCCCCACAGCACC
TACAACCTCTACTGCCCCTTCCGGGCAGAGGAGCGCACGCGGCAGCTCGAGGTG
CTGGCCCAGCAGATTGCCACGCTGTGCGCCACCCTGCAGGAGTACCCGGCCATCC
GCTACCGCAAGGGCCCAGAGGACACAGCCCAGTTGGCCCACGCCGTCCTGGCCA
AGCTGAACGCCTTCAAGGCAGACACTCCCAGTCTGGGCGAGGGCCCAGAGAAAA
CCCGCTCCCAGCTGCTGATAATGGACCGGGCAGCTGACCCCGTGTCCCCACTACT
GCATGAGCTCACGTTCCAGGCCATGGCGTATGATCTGCTGGACATAGAGCAGGA
CACATACAGGTATGAGACCACCGGGCTGAGCGAGGCGCGGGAGAAGGCCGTCTT
GCTGGACGAGGACGATGACTTGTGGGTGGAGCTTCGCCACATGCATATCGCAGA
TGTGTCCAAGAAGGTCACGGAGCTCCTGAGGACCTTCTGTGAGAGCAAGAGGCT
GACCACGGACAAGGCGAACATCAAAGACCTATCCCAGATCCTGAAAAAGATGCC
GCAGTACCAGAAGGAGCTGAATAAGTATTCTACGCACCTGCATCTAGCAGATGA
TTGTATGAAGCACTTCAAGGGCTCGGTGGAGAAGCTGTGTAGTGTGGAGCAGGA
CCTGGCCATGGGCTCCGACGCAGAGGGGGAGAAGATCAAGGACTCCATGAAGCT
GATCGTTCCGGTGCTGCTGGACGCGGCGGTGCCCGCCTACGACAAGATCCGGGT
CCTGCTGCTCTACATCCTCCTTCGGAATGGTGTGAGTGAGGAGAACCTGGCCAAG
CTGATCCAGCATGCCAATGTACAGGCGCACAGCAGCCTCATCCGTAACCTGGAG
CAGCTGGGAGGCACTGTCACCAACCCCGGGGGCTCGGGGACCTCCAGCCGGCTG
GAGCCGAGAGAACGCATGGAGCCCACCTATCAGCTGTCCCGCTGGACCCCGGTC
ATCAAGGATGTAATGGAGGACGCCGTGGAGGACCGGCTGGACAGGAACCTGTGG
CCCTTCGTATCCGACCCCGCCCCCACGGCCAGCTCCCAGGCCGCTGTCAGTGCCC
GCTTCGGTCACTGGCACAAGAACAAGGCTGGCATAGAAGCCCGGGCGGGCCCCC
GGCTCATCGTGTATGTCATGGGCGGTGTGGCCATGTCAGAGATGAGGGCCGCCTA
CGAGGTGACCAGGGCCACCGAGGGCAAGTGGGAGGTGCTCATTGGCTCCTCACA
CATCCTCACCCCGACCCGCTTCCTGGATGACCTGAAGGCACTGGACAAGAAGCT
GGAGGACATTGCCCTGCCCTGA
Wildtype STXBP2 amino acid sequence]
SEQ ID NO: 8
MAPSGLKAVVGEKILSGVIRSVKKDGEWKVLIMDHPSMRILSSCCKMSDILAEGITIV
EDINKRREPIPSLEAIYLLSPTEKAQAQRVIHLPQSVQALIKDFQGTPTFTYKAAHIFFT
DTCPEPLFSELGRSRLAKVVKTLKEIHLAFLPYEAQVFSLDAPHSTYNLYCPFRAEER
TRQLEVLAQQIATLCATLQEYPAIRYRKGPEDTAQLAHAVLAKLNAFKADTPSLGEG
PEKTRSQLLIMDRAADPVSPLLHELTFQAMAYDLLDIEQDTYRYETTGLSEAREKAV
LLDEDDDLWVELRHMHIADVSKKVTELLRTFCESKRLTTDKANIKDLSQILKKMPQY
QKELNKYSTHLHLADDCMKHFKGSVEKLCSVEQDLAMGSDAEGEKIKDSMKLIVPV
LLDAAVPAYDKIRVLLLYILLRNGVSEENLAKLIQHANVQAHSSLIRNLEQLGGTVTN
PGGSGTSSRLEPRERMEPTYQLSRWTPVIKDVMEDAVEDRLDRNLWPFVSDPAPTAS
SQAAVSARFGHWHKNKAGIEARAGPRLIVYVMGGVAMSEMRAAYEVTRATEGKW
EVLIGSSHILTPTRFLDDLKALDKKLEDIALP
Forward primer
SEQ ID NO: 9
TGGAGTGGGACAGAGAAATTAACA
Reverse primer
SEQ ID NO: 10
GCTGGTTTTGCGATTCTTCAA
SEQ ID NO: 11
(gcc)gccRccAUGG
SEQ ID NO: 12
CACCATGGCGG
SEQ ID NO: 13
CGCCATGGCGG
SEQ ID NO: 14
CACGATGGCGG
SEQ ID NO: 15
CACCATGACGG
SEQ ID NO: 16
CGCGATGGCGG
SEQ ID NO: 17
CGCCATGACGG
SEQ ID NO: 18
CACGATGACGG
SEQ ID NO: 19
CGCGATGACGG
SEQ ID NO: 20
GCCACCATGG
SEQ ID NO: 21
ATCGTGAGGCTCCGGTGCCCGTCAGTGGGCAGAGCGCACATCGCCCACAGTCCCCGAGAA
GTTGGGGGGAGGGGTCGGCAATTGAACCGGTGCCTAGAGAAGGTGGCGCGGGGTAAACTG
GGAAAGTGATGTCGTGTACTGGCTCCGCCTTTTTCCCGAGGGTGGGGGAGAACCGTATAT
AAGTGCAGTAGTCGCCGTGAACGTTCTTTTTCGCAACGGGTTTGCCGCCAGAACACAGGT
AAGTGCCGTGTGTGGTTCCCGCGGGCCTGGCCTCTTTACGGGTTATGGCCCTTGCGTGCC
TTGAATTACTTCCACCTGGCTGCAGTACGTGATTCTTGATCCCGAGCTTCGGGTTGGAAG
TGGGTGGGAGAGTTCGAGGCCTTGCGCTTAAGGAGCCCCTTCGCCTCGTGCTTGAGTTGA
GGCCTGGCCTGGGCGCTGGGGCCGCCGCGTGCGAATCTGGTGGCACCTTCGCGCCTGTCT
CGCTGCTTTCGATAAGTCTCTAGCCATTTAAAATTTTTGATGACCTGCTGCGACGCTTTT
TTTCTGGCAAGATAGTCTTGTAAATGCGGGCCAAGATCTGCACACTGGTATTTCGGTTTT
TGGGGCCGCGGGCGGCGACGGGGCCCGTGCGTCCCAGCGCACATGTTCGGCGAGGCGGGG
CCTGCGAGCGCGGCCACCGAGAATCGGACGGGGGTAGTCTCAAGCTGGCCGGCCTGCTCT
GGTGCCTGGCCTCGCGCCGCCGTGTATCGCCCCGCCCTGGGCGGCAAGGCTGGCCCGGTC
GGCACCAGTTGCGTGAGCGGAAAGATGGCCGCTTCCCGGCCCTGCTGCAGGGAGCTCAAA
ATGGAGGACGCGGCGCTCGGGAGAGCGGGCGGGTGAGTCACCCACACAAAGGAAAAGGGC
CTTTCCGTCCTCAGCCGTCGCTTCATGTGACTCCACGGAGTACCGGGCGCCGTCCAGGCA
CCTCGATTAGTTCTCGAGCTTTTGGAGTACGTCGTCTTTAGGTTGGGGGGAGGGGTTTTA
TGCGATGGAGTTTCCCCACACTGAGTGGGTGGAGACTGAAGTTAGGCCAGCTTGGCACTT
GATGTAATTCTCCTTGGAATTTGCCCTTTTTGAGTTTGGATCTTGGTTCATTCTCAAGCC
TCAGACAGTGGTTCAAAGTTTTTTTCTTCCATTTCAGGTGTCGTGAG

1. A nucleic acid sequence comprising an expression-optimized nucleic acid sequence encoding a Munc13-4 polypeptide.

2. The nucleic acid sequence of claim 1, wherein the expression-optimized nucleic acid sequence comprises SEQ ID NO: 1 or a sequence having at least 95% identity to SEQ ID NO: 1.

3. A nucleic acid sequence comprising an expression-optimized nucleic acid sequence encoding a STXBP2 polypeptide.

4. The nucleic acid sequence of claim 3, wherein the expression-optimized nucleic acid sequence comprises SEQ ID NO: 2 or a sequence having at least 95% identity to SEQ ID NO: 2.

5. A polypeptide encoded by the nucleic acid sequence of claim 1.

6. An expression vector comprising the nucleic acid sequence of claim 1.

7. (canceled)

8. (canceled)

9. (canceled)

10. A cell comprising the nucleic acid sequence of claim 1.

11. A cell comprising the vector of claim 6.

12. (canceled)

13. The cell of claim 10, wherein the cell is a hematopoietic stem cell or a hematopoietic stem cell lineage cell.

14. The cell of claim 13, wherein the hematopoietic stem cell lineage cell is a T cell.

15. (canceled)

16. A method of making the cell of claim 10 comprising introducing into the cell a nucleic acid sequence comprising an expression-optimized nucleic acid sequence encoding a Munc13-4 polypeptide or a nucleic acid sequence comprising an expression-optimized nucleic acid sequence encoding a STXBP2 polypeptide.

17. The method of claim 16, wherein the nucleic acid sequence is introduced into the cell by targeted nuclease-mediated insertion of the nucleic acid sequence into the cell.

18. (canceled)

19. (canceled)

20. A cell made by the method of claim 16.

21. A method of treating familial hemophagocytic lymphohistiocytosis (HLH) in a subject comprising:
a. introducing into a population of cells obtained from the subject a nucleic acid sequence comprising an expression-optimized nucleic acid sequence encoding a Munc13-4 polypeptide or a nucleic acid sequence comprising an expression-optimized nucleic acid sequence encoding a STXBP2 polypeptide to provide a population of genetically modified cells; and
b. transplanting the genetically modified cells of step (a) into the subject.

22. The method of claim 21, further comprising culturing the genetically modified cells prior to transplantation into the subject

23. The method of claim 22, wherein culturing comprises conditions for expansion.

24. The method of claim 21, wherein the cells obtained from the subject are hematopoietic stem cells or hematopoietic stem cell lineage cells.

25. The method of claim 24, wherein the cells obtained from the subject are hematopoietic stems cells and wherein the culturing comprises conditions that promote differentiation of the hematopoietic stem cells into T cells.

26. (canceled)

27. (canceled)

28. The method of claim 21, wherein the cells obtained from the subject are endothelial cells.

29. The method of claim 21, further comprising in vivo administration of an expression optimized nucleic acid sequence encoding a Munc13-4 or STXB2 polypeptide to the subject.

30. The method of claim 29, wherein non-hematopoietic cells are modified in the subject.

31. (canceled)

32. A method for treating HLH in a subject comprising administering to the subject a nucleic acid sequence comprising an expression-optimized nucleic acid sequence encoding a Munc13-4 polypeptide or a nucleic acid sequence comprising an expression-optimized nucleic acid sequence encoding a STXBP2 polypeptide.

33. The method of claim 32, wherein the nucleic acid is administered to the subject in a vector.

34. The method of claim 33, wherein the vector is a viral vector.

35. The method of claim 34, wherein the vector is an adeno-associated viral vector, an adenoviral vector, a lentiviral vector or a retroviral vector.

36. The method of claim 32, wherein non-hematopoietic stem cells are modified in the subject.

37. The method of claim 32, wherein a hematopoietic stem cell lineage cells are modified in the subject.

38. (canceled)

39. (canceled)

40. (canceled)

41. (canceled)
